(19) United States
(12) Patent Application Publication
(10) Pub. No.: US 2021/0187014 A1
(43) Pub. Date: Jun. 24, 2021

(54) Methods, Systems And Compositions For The Novel Use of Enterobactin to Treat Iron Deficiency And Related Anemia And Promote Red Blood Cell Production

(71) Applicant: The Regents of the University of Colorado, a body corporate, Denver, CO (US)

(72) Inventors: Min Han, Boulder, CO (US); Bin Qi, Boulder, CO (US); Mingxue Cui, Boulder, CO (US); Aileen K. Sewell, Longmont, CO (US)

(21) Appl. No.: 17/151,530

(22) Filed: Jan. 18, 2021

Related U.S. Application Data
(63) Continuation-in-part of application No. PCT/US2019/042425, filed on Jul. 18, 2019.
(60) Provisional application No. 62/700,480, filed on Jul. 19, 2018.

(51) Int. Cl. A61K 33/26; A61K 35/741; A23L 33/135; A61P 7/06

(52) U.S. Cl. CPC A61K 33/26; A61P 7/06; A23L 33/135; A61K 35/741

(57) ABSTRACT

In one embodiment, the invention relates to the use of ferric enterobactin (Fe Ent), and/or Fe-Ent analogs as a therapeutic agent to treat iron deficiency and anemia. In a preferred embodiment, Fe-Ent and/or Fe-Ent analogs may be delivered to a host organism, such as a human subject to treat an iron-related disease condition or other anemia. In alternative embodiments, delivered to a host organism, such as a human subject to promote production or red blood cells. In such an embodiment, Ent and/or Ent analogs may be delivered to a human subject in need thereof through a pharmaceutical composition or through a genetically engineered bacteria, such as a probiotic organism configured to express Ent.

### CROSS-REFERENCE TO RELATED APPLICATIONS

This U.S. Continuation-in-part claims the benefit of and priority to International PCT Application No. PCT/US2019/042425, filed Jul. 18, 2019, which claims the benefit of and priority to U.S. Provisional Application No. 62/700,480, filed Jul. 19, 2018. The entire specification and figures of the above-referenced applications are hereby incorporated, in its entirety by reference.

### SEQUENCE LISTING

The instant application contains a Sequence Listing which has been submitted electronically in ASCII format and is hereby incorporated by reference in its entirety. Said ASCII copy, created on January 13, 2021, is named “90245.00072-Seq-Listing-AF.txt” and is 25.9 Kbytes in size.

### TECHNICAL FIELD

The present invention relates the novel benefits of individual microbiota-derived molecules in host animals. For example, the bacteria-secreted enterobactin (Ent) is an iron scavenging siderophore with presumed negative effects on hosts. However, the high prevalence of Ent-producing commensal bacteria in human gut indicates a potential host mechanism to beneficially use Ent to treat disease conditions related to iron metabolism, and in particular iron deficiencies. Through a novel and unique assay, the present inventors discovered an unexpected and striking role of Ent in supporting growth and the labile iron pool in an exemplary eukaryotic host organism. The present inventors demonstrated that Ent promotes mitochondrial iron uptake and does so, surprisingly, by binding to the ATP synthase α-subunit, which acts inside of mitochondria and independently of ATP synthase. The present inventors also demonstrated the conservation of this mechanism in mammalian cells. This study reveals a new paradigm for the “iron tug-of-war” between commensal bacteria and their hosts and an important mechanism for mitochondrial iron uptake and homeostasis.

### BACKGROUND OF THE INVENTION

Iron deficiency is the most prevalent nutrient-deficiency disorder and the most common cause of anemia, affecting >¼ of the global population, particularly women and children, based on the analyses by the World Health Organization (WHO) and other literature (Stevens et al., 2013; WHO, 2015). Anemia is also responsible for about 9% of global disability. Importantly, the primary treatment of this disorder, oral iron supplementation, has serious problems. First, oral iron supplementation has very low efficacy, partly because it induces hormonal changes (increased hepcidin) that block iron uptake (Muckenthaler et al., 2017),(Cook and Reddy, 1995; Moretti et al., 2015). Second, this treatment has well-known adverse side effects that may lead to increased mortality, especially among people with other diseases (Sazawal et al., 2006). For examples, oral iron supplementation is known to promote inflammation through inducing free radicals and unfavorable changes to human microbiota composition that are likely causal to some of the adverse side effects (Jaeggi et al., 2015; Kortman et al., 2015; Lund et al., 1999; Tang et al., 2017). It is also known that anemia is highly prevalent among people with common GI diseases such as Inflammatory Bowel Diseases (IBD) (>50% IBD patients are also anemic in the US (Koutroubakis et al., 2015). Many anemia patients, including those suffering from other GI diseases, are totally intolerable to oral iron supplementation, requiring intravenous infusion that is also well known for association with health risks and side effects (Auerbach and Macdougall, 2017; Munoz et al., 2009). Defects in iron trafficking systems are causal to certain iron-deficiency anemia (Brissot et al., 2011), and increased iron uptake efficiency may be key to a transformative treatment for most anemic patients.

As detailed below, the present inventors demonstrate that Enterobactin (Ent) has the potential to increase the efficiency of iron absorption without high-level oral iron supplementation and its associated side effects. Indeed, the unanticipated clinical significance of this finding was recognized in commentaries by experts in top journals (Anderson, 2018). For example, an article in the New England Journal of Medicine (NEJM) in November 2018, highlighted the novel and unexpected clinical implications of the inventors' basic research and highlighted the predicted ability of Ent to iron into mitochondria in several cell types in humans (Gregory Anderson: “Iron Wars—The Host Strikes Back” NEJM 2018) (FIG. 17). Notably, Ent is a catecholate siderophore produced almost exclusively by enterobacteria to scavenge iron from the environment. The scavenging role of Ent is expected to have a negative impact on iron homeostasis and certain cellular processes in the host, given that siderophores are known to be key virulence mediators of pathogens. In order to inhibit the growth of pathogenic bacteria that rely on Ent to scavenge iron from host cells, the mammalian immune system produces the Ent-binding protein lipocalin 2 that sequesters Ent. Such a defense system may have negative effects on the host iron pool and animal functions. More importantly, this mechanism does not explain how host animals would cope with abundant Ent from non-infectious gut microbiota, of which enterobacteria are the most prevalent commensal microbes in both human and C. elegans. Given the symbiotic relationship between commensal E. coli and host, there may exist an unknown beneficial mechanism that has evolved in animals to use bacterial Ent for host iron homeostasis.

Iron transport into mitochondria is a key event in iron homeostasis, as much of the cellular labile iron poor is transported into mitochondria to be incorporated into heme and Fe-S complexes (Muckenthaler et al., 2017). In humans, under normal conditions, ˜70% of iron is found in hemoglobin (Zhang and Enns, 2009). Since the iron-binding step of heme biosynthesis occurs in mitochondria, iron transport into mitochondria is critical to hemoglobin production or erythropoiesis. Under anemic conditions where the hemoglobin count is low, an even higher percentage of iron needs to be transported into mitochondria.

Indeed, there may be several beneficial effects of supplemented Ent being used by bacteria. In one embodiment, this may include having more Ent might boost the prevalence of Ent-utilizing bacteria (mainly E. coli in humans), which may have a positive therapeutic effect in some instances. In fact, having too little commensal E. coli might contribute to anemia or other unhealthy conditions. The prevalence of commensal E. coli varies across populations. In another example, it is now well known that changes in gut microbiota composition (to unhealthy ones) is a very significant, negative side effect of taking oral iron supplements. If taking Ent permits dramatic reduction in the effective iron supplement dose, patients are more likely to obtain strong overall benefits by potential minimizing the unfavorable changes in gut microbiota composition.

Additionally, as part of the mammalian host immune response, Lipocalin 2 expression is induced by pathogen attack and is known to bind to Ent, with the goal to sequester Ent and its bound iron from benefiting the proliferation of certain infectious bacteria. Therefore, adding more Ent under this infection condition might interfere with the role of lipocalin 2 in combating these bacteria. However, this concern may not be a serious obstacle to the potential therapeutic usage of Ent to treat anemia. First, since the anti-infection effect of lipocalin 2 is really to create a low iron environment for bacteria, taking high levels of iron by oral supplementation would probably have a stronger effect to benefit iron-hungry infectious bacteria than Ent would in compromising the plan by Lipocalin2 in anemic patients. We need to remember that bacteria can uptake iron through Ent-independent ways, and that bacteria only need Ent under low iron conditions. Therefore, the potential side effect of Ent in this regard already exists in anemic patients who need to take oral iron.

Thus, there remains a substantial need in the art for the identification and characterization of the molecular components and host interactions involved in bacterial Ent molecular biology. In particular, there exists a need for novel systems, methods and compositions for the development of Ent-based therapies and pharmaceutical compositions that may be used to regulate and treat iron-related disease conditions in animals and humans.

### SUMMARY OF THE INVENTION(S)

In one embodiment, the present inventors demonstrate a unique and sensitive assay to test the impact of E. coli genes on animal development. In particular, the present inventors identified a new paradigm regarding the effect of a siderophore (enterobactin, or Ent) produced by commensal bacteria on host physiology. The present inventors discovered that Ent promotes mitochondrial iron level in host animals and beneficially impacts host development. Ent executes this function by binding to the a-subunit of the host mitochondrial ATP synthase, and this binding is independent of the whole ATP synthase complex. This previously unknown mechanism may counteract the scavenging role of bacterial Ent and its impact on the host labile iron pool (FIG. 7) and this function should enhance the symbiotic relationship between microbes and animals. Due to the conservation of this function between C. elegans and humans, the present invention may be consistent with the high prevalence of enterobacteria in the gut of both animal species and the ability of commensal E. coli in mammals to produce Ent. This novel inventive technology presents a new paradigm regarding the competition (“tug-of-war” for iron) between microbes and host cells, which is distinct from the function of the well-studied mammalian lipocalin 2 that binds to Ent as a defense mechanism against pathogenic bacteria (FIG. 7).

Iron deficiency is one of the most prevalent nutritional disorders that threaten the health of a large population of children and women in the world (WHO, 2002). The composition and behavior of the human gut microbiome, that produces various iron binding siderophores, may have high impacts on the genesis and treatment of this disorder. The present invention demonstrates that Ent, and in particular Ent-Fe supplementation in animal and human models, facilitates iron uptake and growth under both low and high iron conditions, which may in turn suggest that iron level in the gut does not usually reach the height leading to a full repression of Ent production from the gut microbiota. The profound impact of additional Ent-Fe supplementation on iron level and animal growth under iron-deficient condition (FIG. 2G) suggests that disruption of microbiotal composition may significantly contribute to human iron deficiency disorder and, as one specific embodiment of the current invention, the potential of Ent-Fe supplementation as a treatment to this widely spread human health problem.

The present inventors have also provided experimental evidence that the ATP synthase α-subunit is not likely to facilitate the mitochondrial iron uptake by a co-transportation model, where Ent-Fe may simply take a ride when the ATP synthase α-subunit is transported into mitochondria. Instead, in one embodiment of the present invention demonstrates a retention model of transport, where the ATP synthase a-subunit inside the mitochondria binds and retains Ent-Fe, implying that Ent-Fe may enter and exit mitochondria through a passive mechanism or a system involving protein transporter. A recent study in mammalian cells suggested that Ent could enter mammalian cells by permeation, while studies in yeast have indicated a transporter that can traffic Ent into fungi cells. In one embodiment, under the retention model, a passive diffusion seems to be more straightforward since Ent-Fe would also exit mitochondria without the interaction with ATP synthase a-subunit.

The present inventive technology further demonstrates that the role of the ATP synthase a-subunit in mitochondrial iron retention is likely independent of the ATP synthase; it neither requires the interaction with other subunits nor its enzymatic activity. Therefore, Ent-Fe³+ may be able to interact with the ATP synthase α-subunit that localizes within the mitochondria but is physically detached from the ATP synthase. However, in one embodiment it may be that the α-subunit localizes at sites away from the ATP synthase in wild type animals, and as a result Ent may still bind to the α-subunit that is associated with ATP synthase under normal conditions, even though Ent-Fe may be capable of interacting with the a-subunit in the absence of the β-subunit.

Iron uptake into mitochondria critically contributes to the regulation of the labile iron pool level, but the mechanism of this process remains to be understood. In the present inventors analyses of C. elegans, the impacts of Ent and ATP-1 on labile iron level are quite profound (FIGS. 2 and 4). These impacts indicate that this newly discovered system involving Ent and the ATP synthase α-subunit represents an important mechanism underlying iron uptake into mitochondria, knowing that Ent-producing enterobacteria are the most prevalent commensal microbes in both C. elegans and humans. In addition, certain embodiments of the inventive technology described herein may indicate a mechanism that could have a significant influence on our understanding of other systems involved in iron trafficking into mitochondria. For example, the retention model might also be involved in the functions of other iron carriers, including a mammalian siderophore. The inventive technology described herein further demonstrates the value of using C. elegans to study the impact of individual microbiota-generated metabolites on host physiology and the symbiotic relationship between animals and gut microbes, as well as provides for therapeutic supplementation of Ent in human and animals that may suffer from or be at risk for iron-deficiency relates disease conditions.

As such, the present invention identifies and characterizes bacterial Ent as a major regulator of iron levels and growth in animals. One aim of the current invention may include systems, methods, and compositions for a novel assay in an exemplary eukaryotic model organism, in this case C. elegans, that elucidates the role of bacterial Ent in supporting animal growth and iron level(s).

Another aspect of the current invention includes systems, methods, and compositions demonstrating an interaction between Ent and the ATP synthase α subunit which facilitates mitochondrial iron uptake both in C. elegans and mammals.

Yet another aspect of the current invention includes the use of Ent promotes host iron homeostasis through its interaction with the ATP synthase α-subunit. In this preferred embodiment, Ent-Fe supplementation may be utilized as a therapeutic agent to treat one or more iron-deficiency related disease conditions. In this preferred embodiment, a therapeutically effective amount of Ent-Fe supplementation may be introduced to a subject in need thereof. The delivery of Ent-Fe may be through pharmaceutical compositions, or even through the introduction of genetically engineered probiotic and/or symbiotic bacteria configured to produce/overproduce exogenous, modified and/or endogenous Ent-Fe.

Another aspect of the invention may further include systems, methods and compositions of treating iron deficiency related conditions and their related anemia. In one preferred embodiment, such systems, methods and compositions treating and/or prophylactically preventing iron deficiency related conditions and their related anemia may include Ent-Fe supplementation as described herein.

Additional aspects of the current invention may include systems, methods and compositions for the use of Ent-Fe and/or ATP-1 as bio-markers of iron-related disease conditions, as well as a diagnostic bio-marker for diagnosing an iron-deficiency related disease condition, and/or a subject's susceptibility to an iron-deficiency related disease condition.

Additional aspects of the invention may include methods and compositions for promoting the production of red blood cells (erythrocytes). In one preferred aspect, a therapeutically effective amount of ferric enterobactin (Fe-Ent), or an Fe-Ent analog, or a pharmaceutically acceptable to salt may be administered to a subject, wherein said Fe-Ent, or analog promoted production of erythrocytes, for example by increasing differentiation of murine erythroid precursor cells into erythrocytes, which may be a treatment for any number of anemia-related conditions. In a preferred aspect, Fe-Ent, or Fe-Ent analog supplementation may be used to treat iron-deficiency, anemia, or may be used for therapeutic uses where a patient may benefit from increase red blood cells to help, for example to oxygenate the blood, heal from a wound of sickness, increase stamina, avoid blood transfusion, aplastic anemic, cancer. In other embodiment, Fe-Ent, or Fe-Ent analog supplementation may be used to counter act drugs or other compounds that may inhibit or reduce red blood cell production, such as drugs for HIV, cancer and other conditions.

In other embodiment, this aspect of the invention may be use to increase the health or performance of a subject. For example, Fe-Ent, or Fe-Ent analog supplementation may increase red blood cells, which may be a treatment for anemia. As used herein, “anemia” refers to a condition whereby the body has fewer than necessary red blood cells thereby resulting in reduced oxygen to cells and tissues. Anemias may be caused by any of several disorders and include but are not limited to anemia due to B12 deficiency, anemia due to folate deficiency, anemia due to iron deficiency, hemolytic anemia, hemolytic anemia due to G-6-PD deficiency, idiopathic aplastic anemia, idiopathic autoimmune hemolytic anemia, immune hemolytic anemia, iegaloblastic anemia, pernicious anemia, secondary aplastic anemia, and sickle cell anemia. Certain symptoms are associated with anemia and include pale skin, dizziness, fatigue, headaches, irritability, low body temperature, numb/cold hands or feet, rapid heartbeat, shortness of breath, weakness and chest pain any of which may be ameliorated by administration of Fe-Ent or Fe-analogs.

Additional aspect of the invention may include one or more of the following embodiments. The present application refers to various journal articles, and other publications, all of which are incorporated herein by reference. The details of one or more embodiments of the invention are set forth herein. Other features, objects, and advantages of the invention will be apparent from the Detailed Description, the FIG.s, the Examples, and the Claims.

### BRIEF DESCRIPTION OF THE FIGURES

The above and other aspects, features, and advantages of the present disclosure will be better understood from the following detailed descriptions taken in conjunction with the accompanying figures, all of which are given by way of illustration only, and are not limiting the presently disclosed embodiments, in which:

FIG. 1. Microbial metabolite enterobactin (Ent) supports C. elegans development. (FIG. 1A) Cartoon diagram, microscope images and bar graph showing that a trace amount of live bacteria supports the postembryonic growth of worms fed heat-killed E. coli. The volume of the worms was measured 4 days after larvae were placed on the plates. (FIG. 1B-C) A bacterial mutant screen identified 5 genes in the enterobactin (Ent) biosynthesis pathway that support host development (as indicated by decreased worm body volume) when fed each of these 5 mutants under the assay condition. Enzymes in red (FIG. 1C) were identified in the screen (FIG. 1B). (FIG. 1D) The growth defect caused by feeding entA- or entF-live E. coli mutants, along with heat-killed E. coli, was fully suppressed by dietary supplementation with Ent. Representative microscope images are shown in FIG. 8A. (FIG. 1E) Supplementation with 2,3-DHBA rescued the growth of worms fed entA-, but not entF-mutant bacteria, confirming that only the final product, Ent, is beneficial for worm growth. (FIG. 1F) The growth defect caused by feeding ent-live E. coli mutants along with heat-killed E. coli was not phenocopied by mutation of fepA, the gene that encodes the E. coli ferric Ent receptor, indicating that Ent does not benefit worm growth through its role in bacterial iron scavenging. Also see FIG. 8B and 8C for role of fepA and worm images. (FIG. 1G) Supplementation with other siderophores (pyoverdine or ferrichrome) did not rescue growth of worms fed entF-mutant along with heat-killed food. Toxicity tests of these siderophores are shown in FIG. 8F-H. (FIG. 1H) CAS staining results of whole worm lysates showing that Ent level in worms fed entF-mutant bacteria is significantly lower than that in worms fed wild-type bacteria. (FIG. 1I) Cartoon diagram of feeding condition, bar graph and statistical analysis showing that worm larvae fed live entA- or entF-E. coli strains alone (bacterial lawn) displayed reduced growth rate compared to worms fed parental wild-type E. coli, indicating a significant benefit from Ent to the host development, even though it was not absolutely required under this feeding condition. For all panels, “n”=number of worms scored. Data are represented as mean±SEM. ***P<0.001. All data are representative of at least three independent experiments.

FIG. 2. Bacterial Enterobactin promotes host iron pool level. (FIG. 2A-E) Cartoon diagrams of feeding conditions, fluorescence images and bar graphs depicting the impact of feeding conditions on host iron level and pftn-2::GFP expression. (FIG. 1A) Worms fed entA- or entF-E. coli with heat-killed E. coli exhibited a dramatic increase in calcein-AM fluorescence (that indicates a decrease in the labile iron level), and this change was fully suppressed by dietary supplementation of Ent. (FIG. 2B) The expression of the iron responsive reporter pftn-2::GFP was decreased in worms fed entA- or entF-E. coli combined with heat-killed E. coli. (FIG. 2C and D) Ent supplementation to heat-killed E. coli recovered the iron level (indicated by both Calcein AM fluorescence and pftn-2::GFP) in growth-arrested worms without rescuing growth, indicating that the Ent effect on the host iron pool in (FIG. 2A) was not likely due an indirect effect of the worm's slower growth rate. (FIG. 2E) Calcein-AM fluorescence intensity is increased in worms fed only entA- or entF-live bacteria, indicating that the benefit of Ent to host iron level increase is not limited to the feeding condition diagramed in

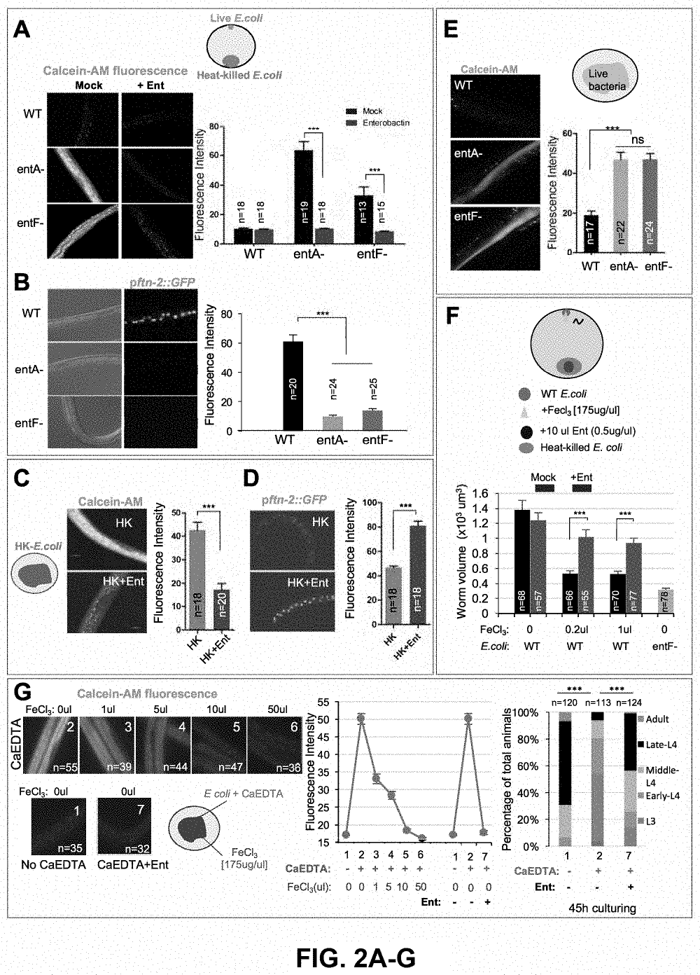

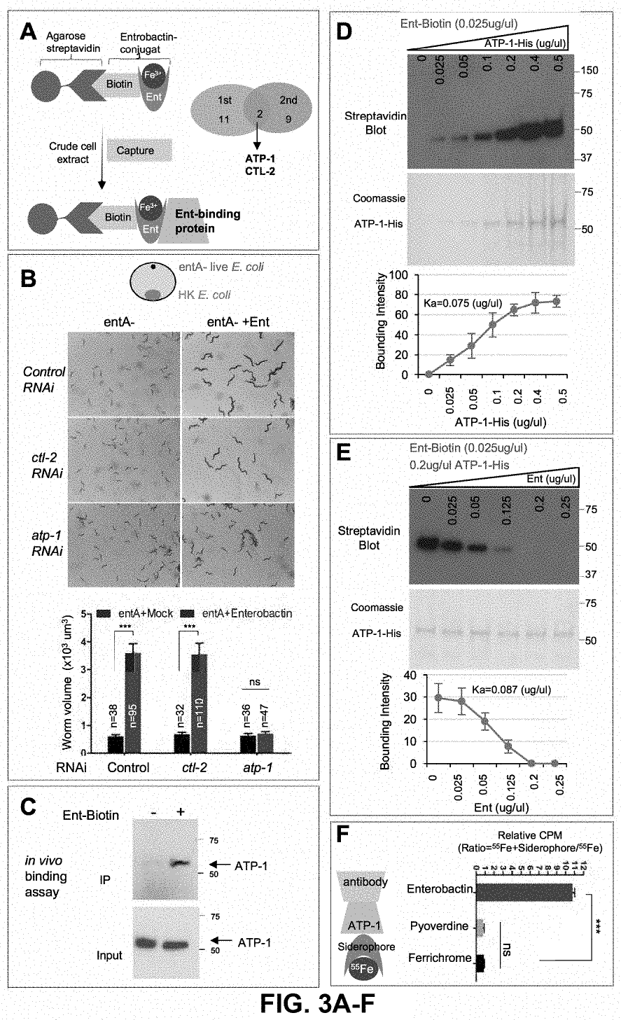

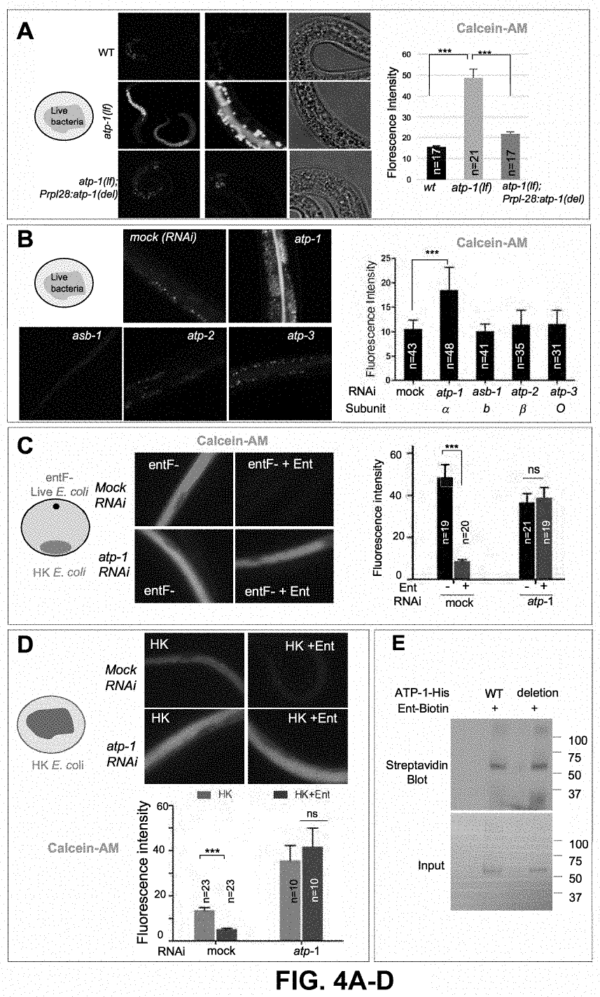

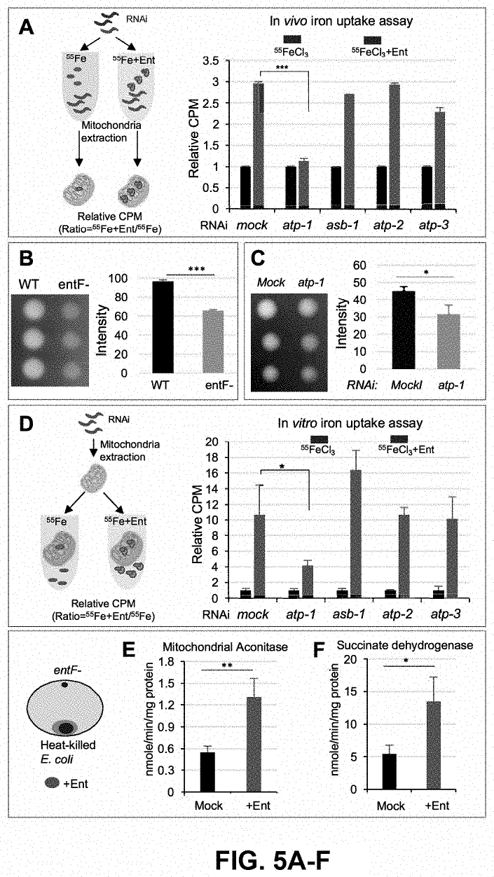

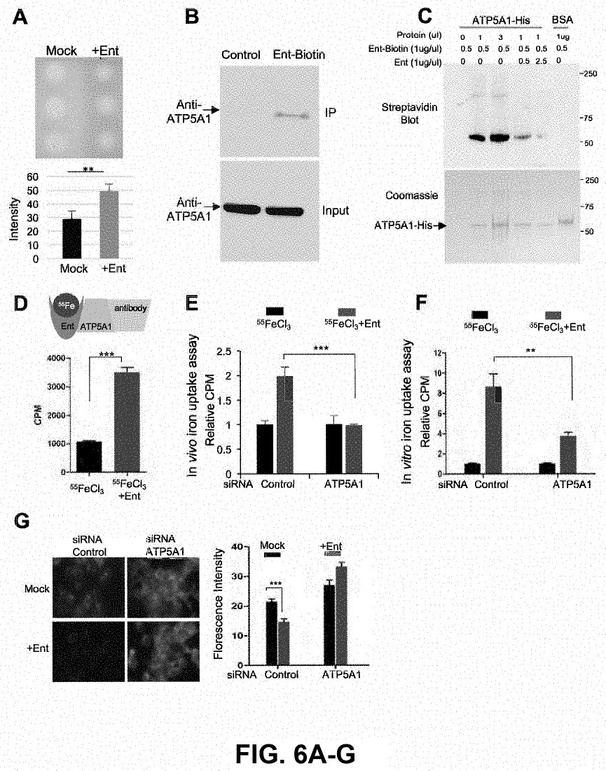

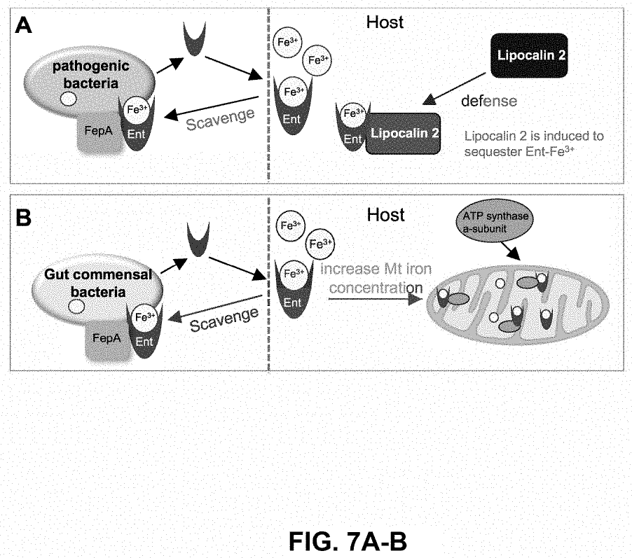

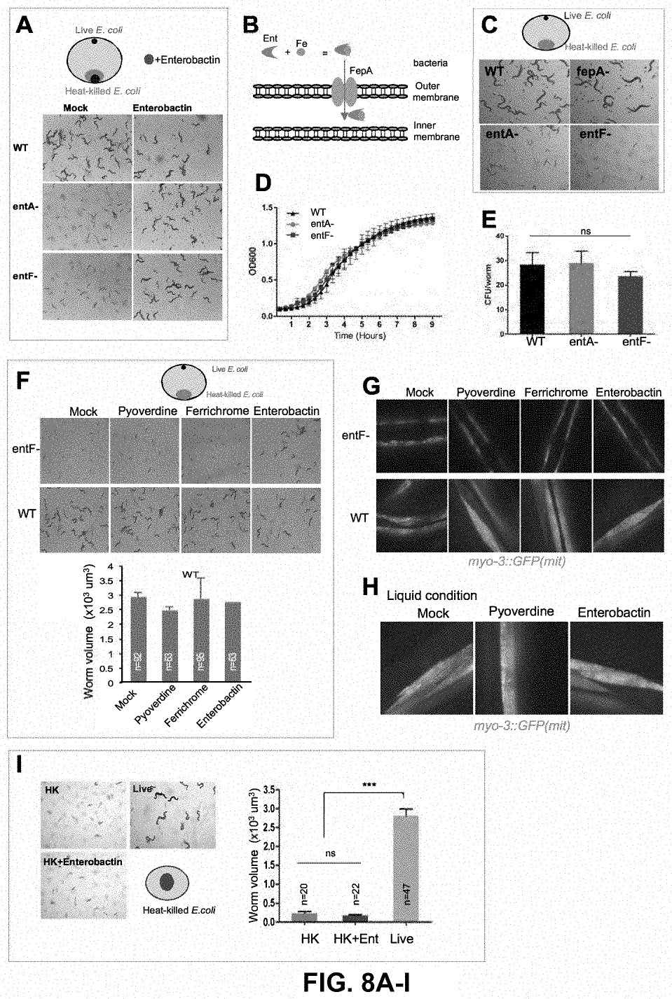

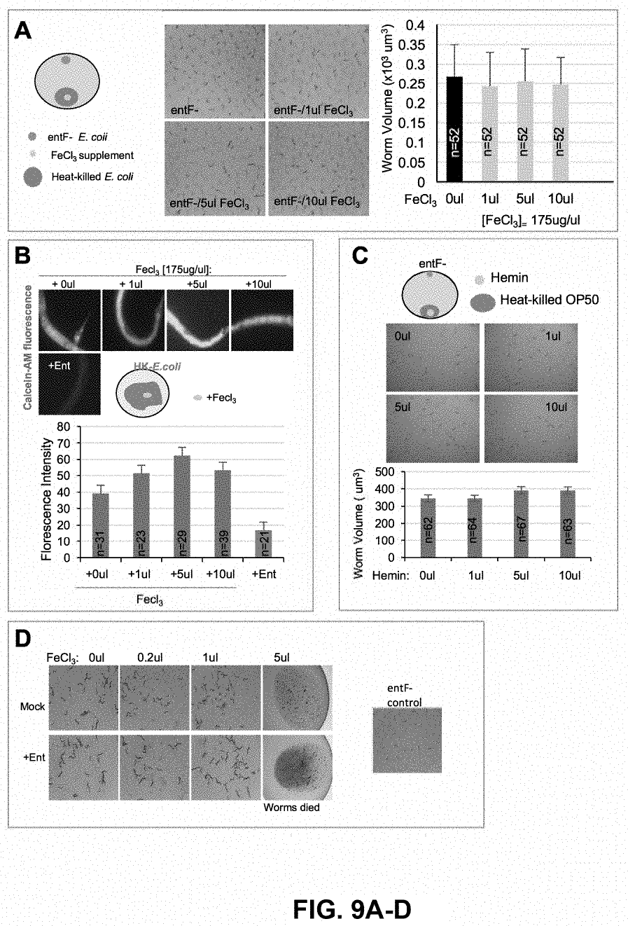

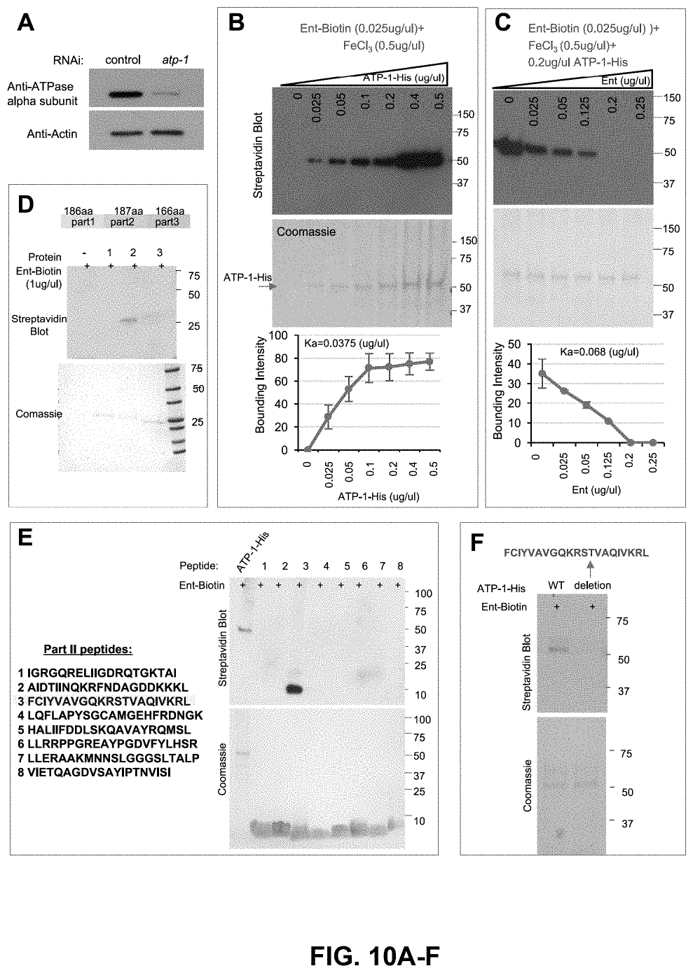

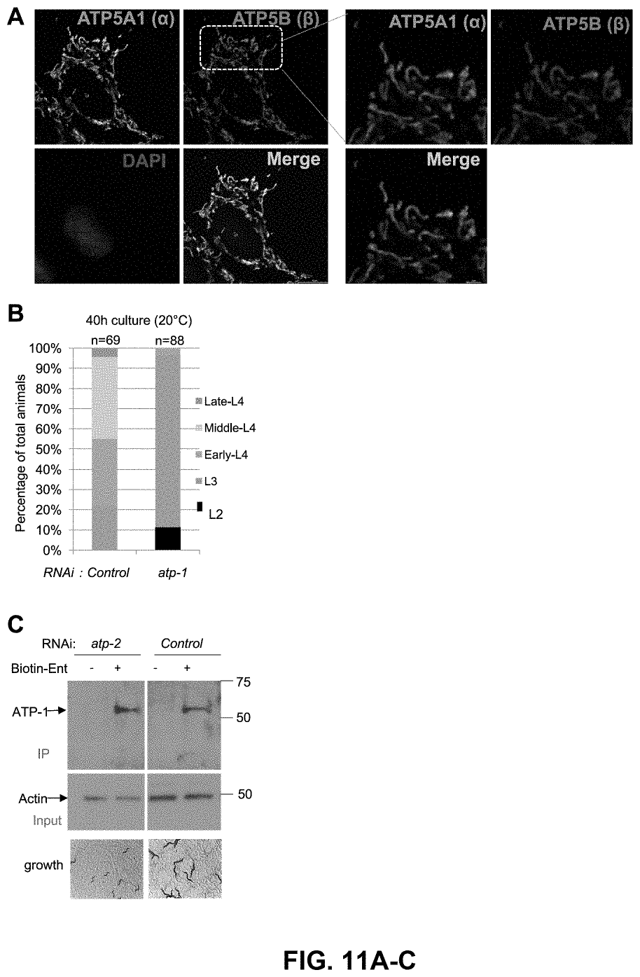

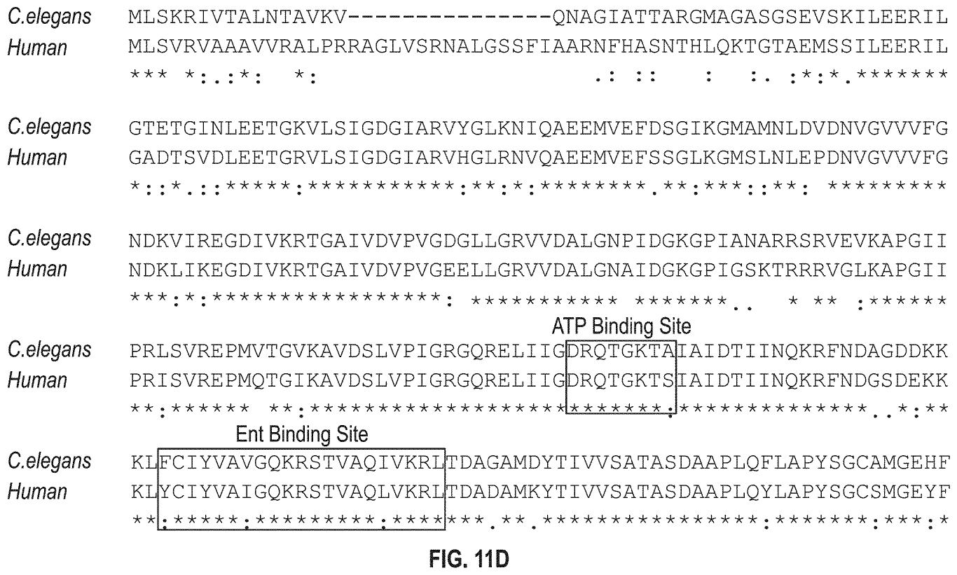

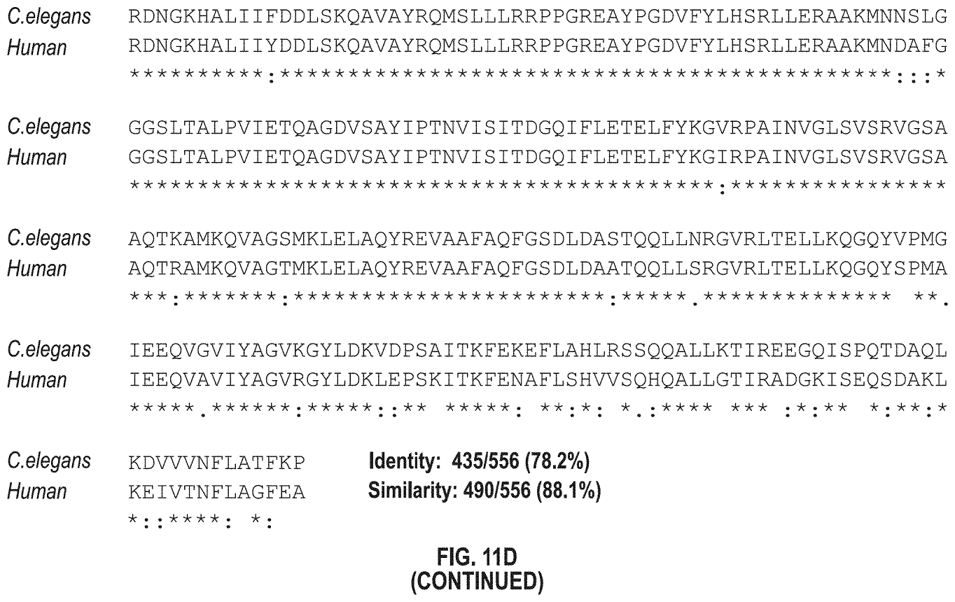

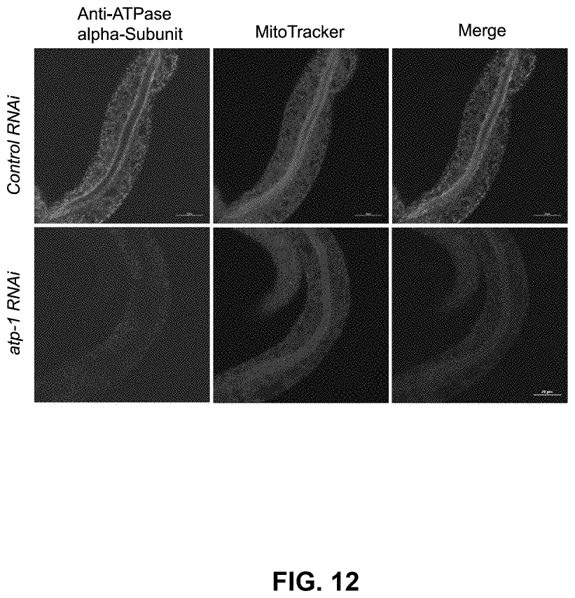

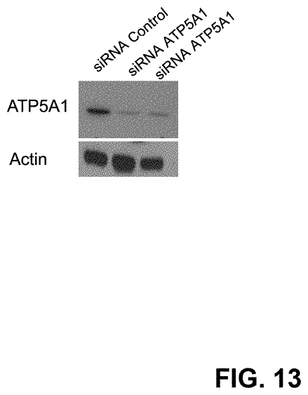

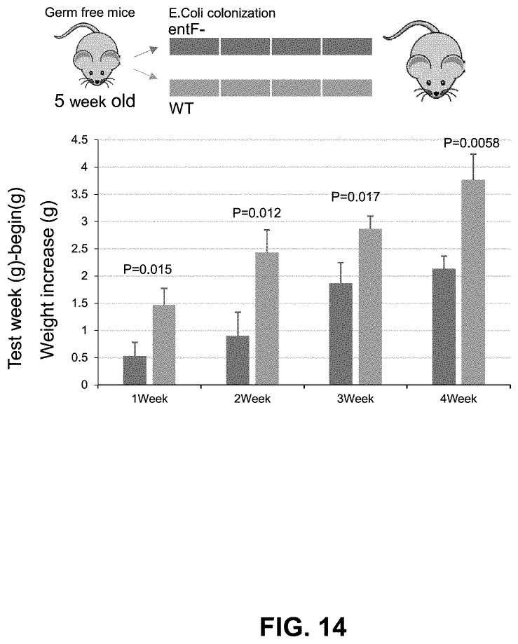

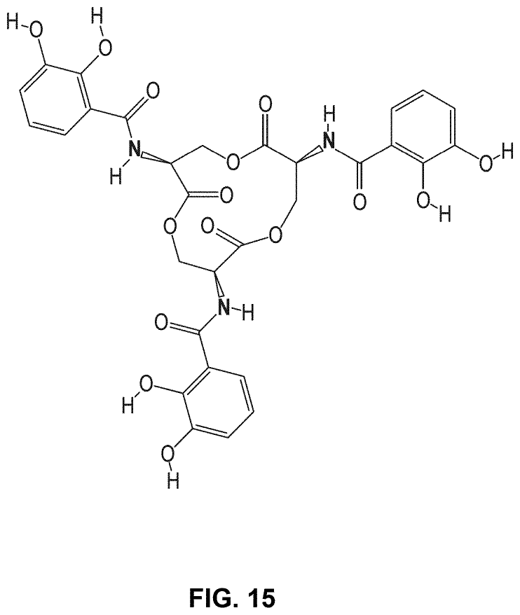

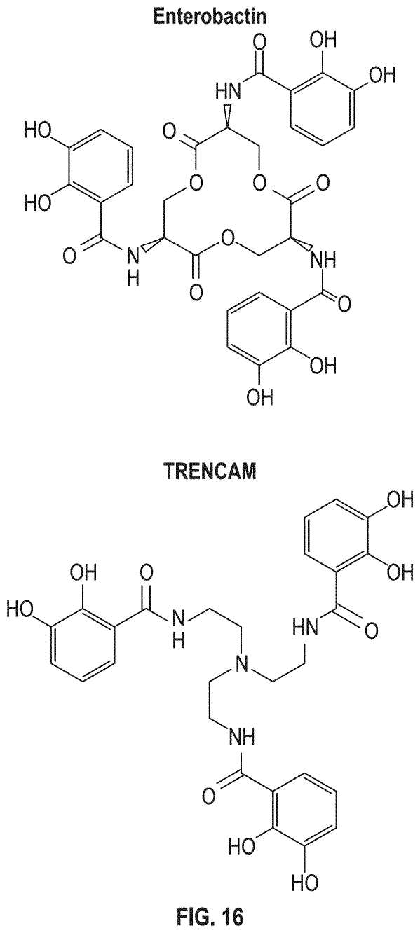

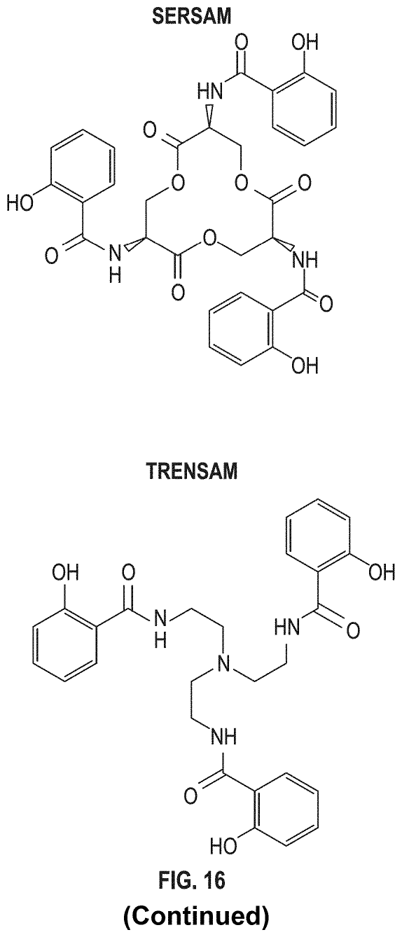

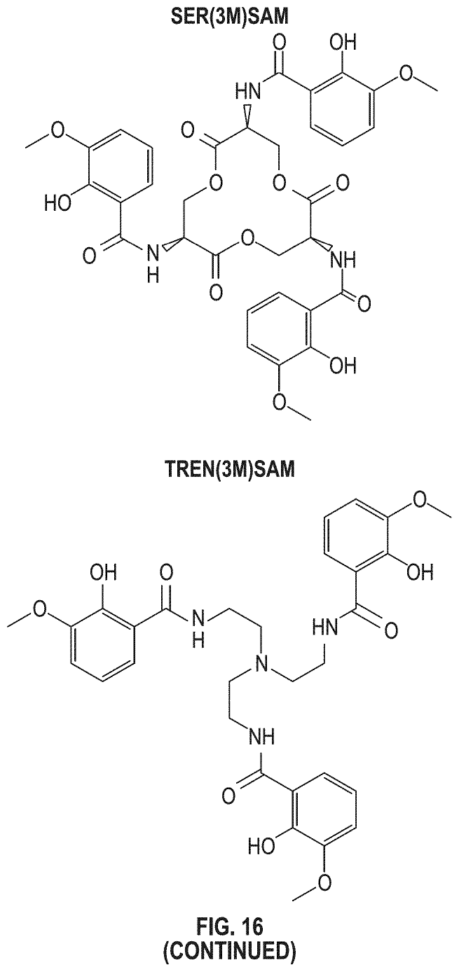

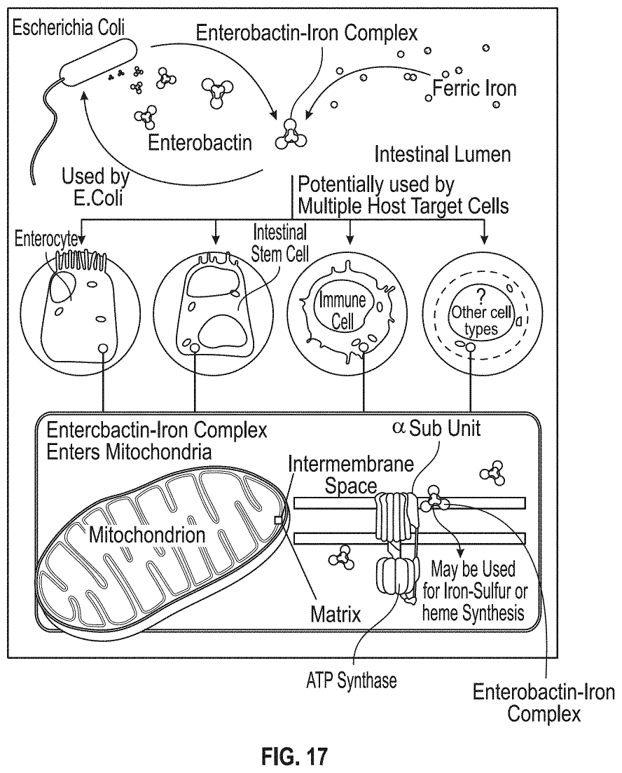

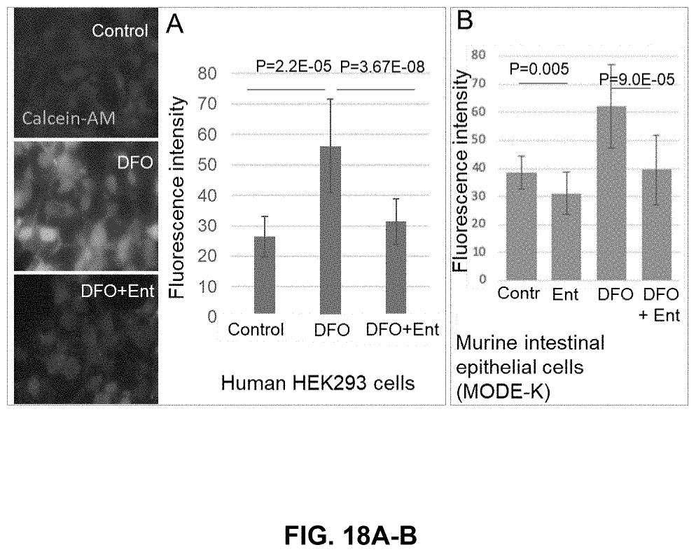

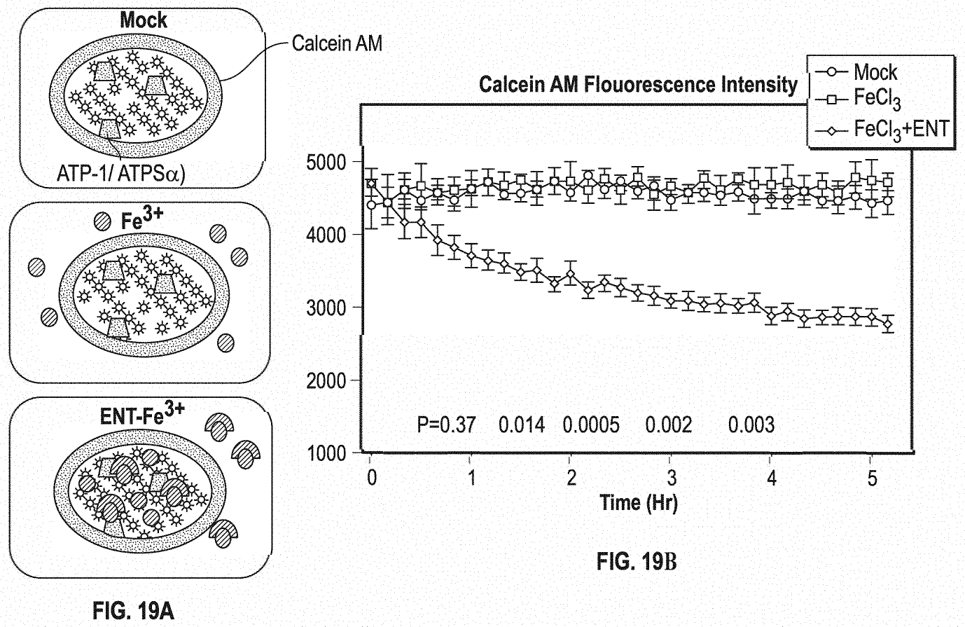

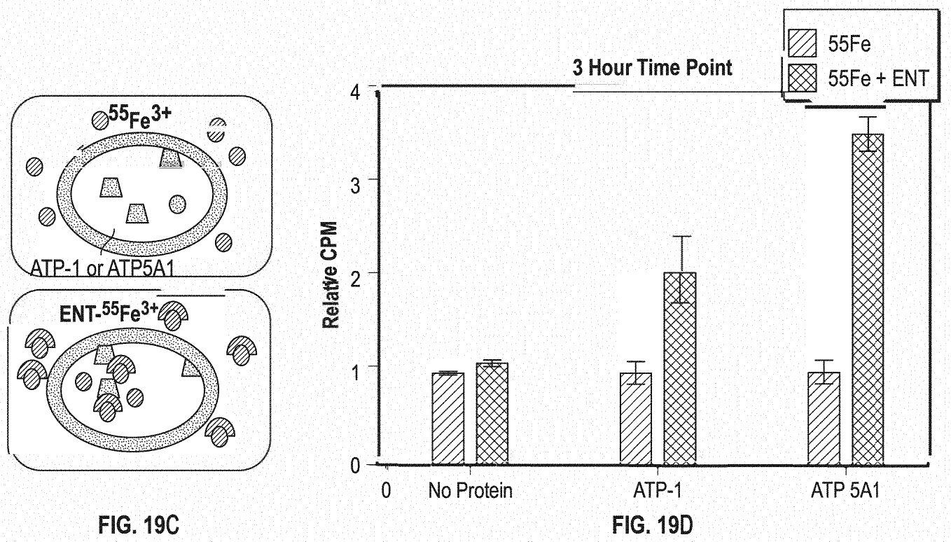

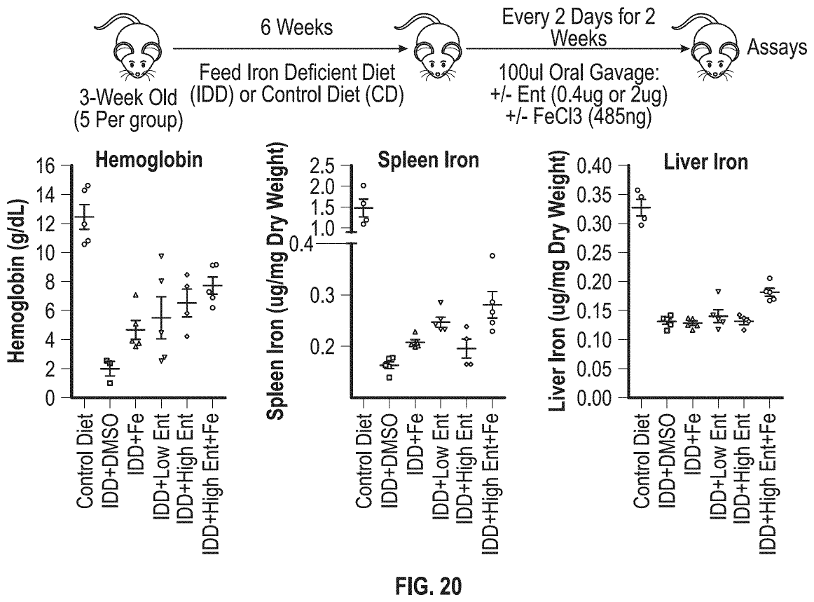

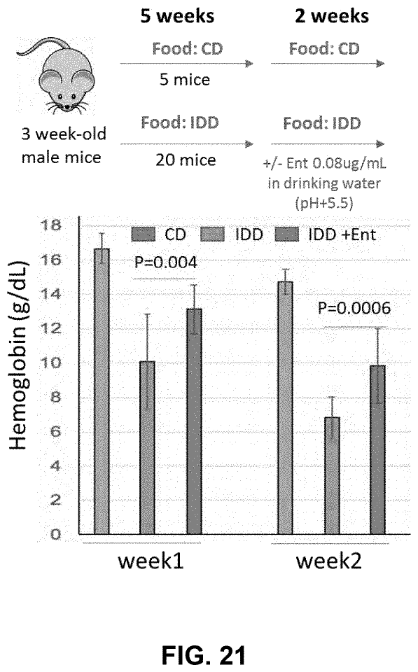

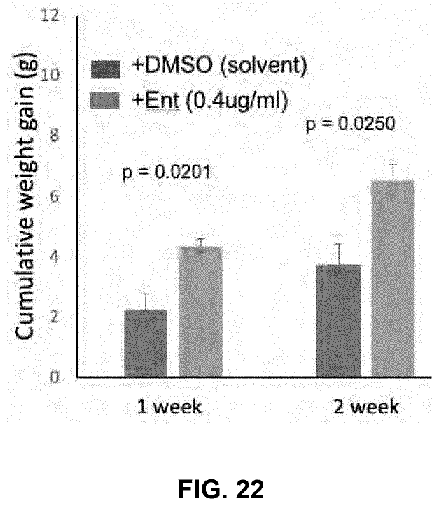

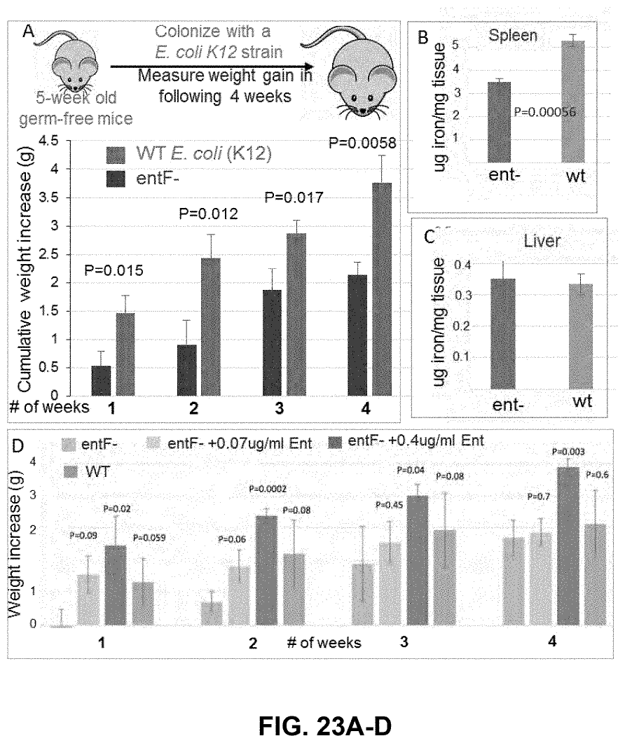

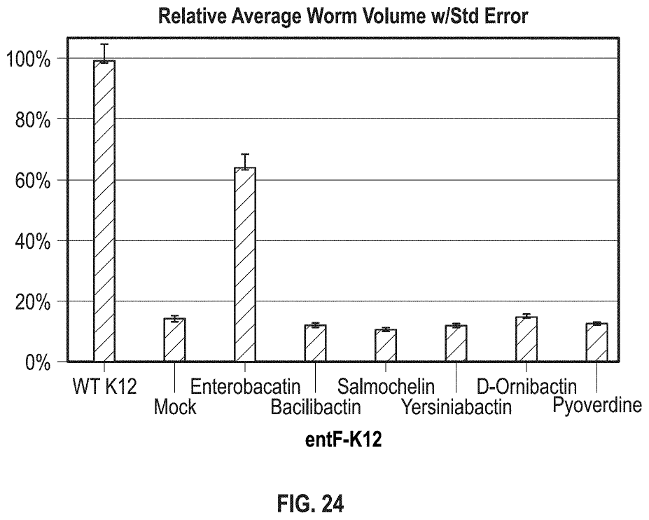

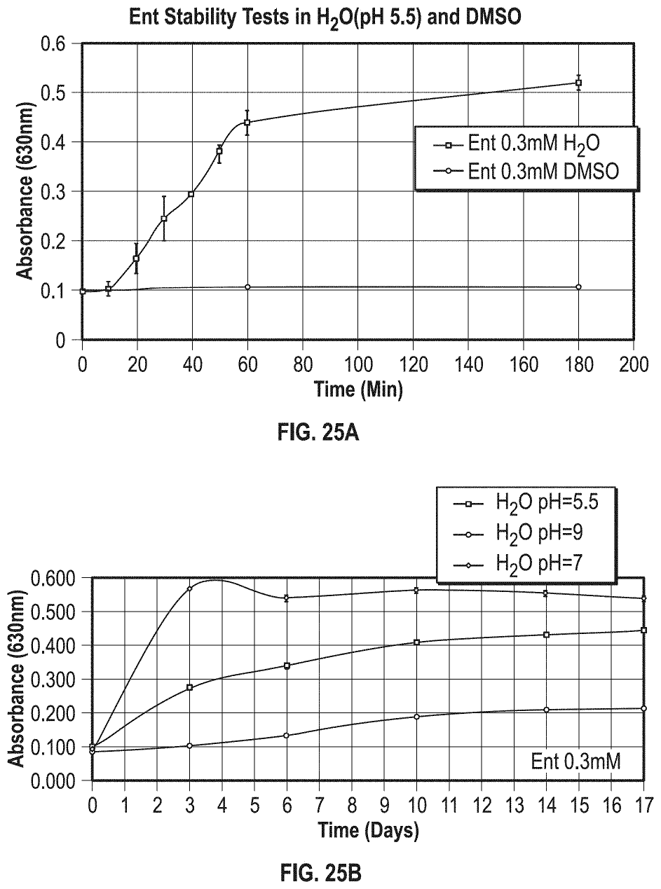

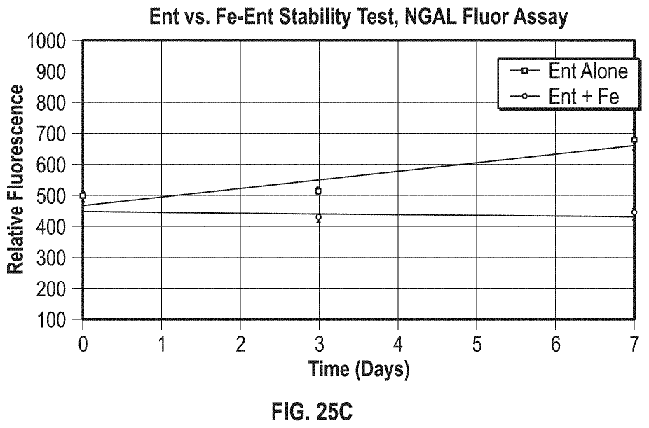

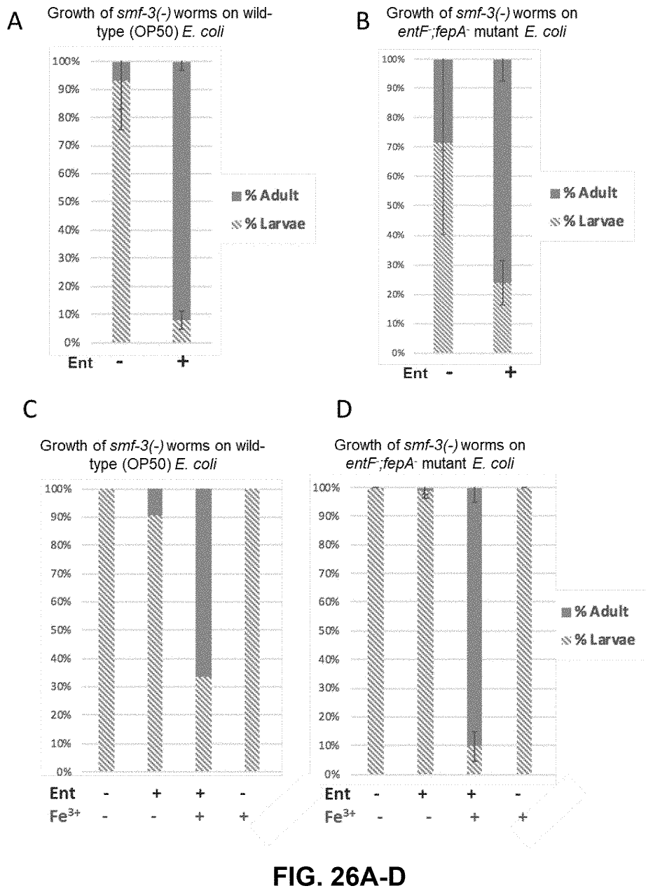

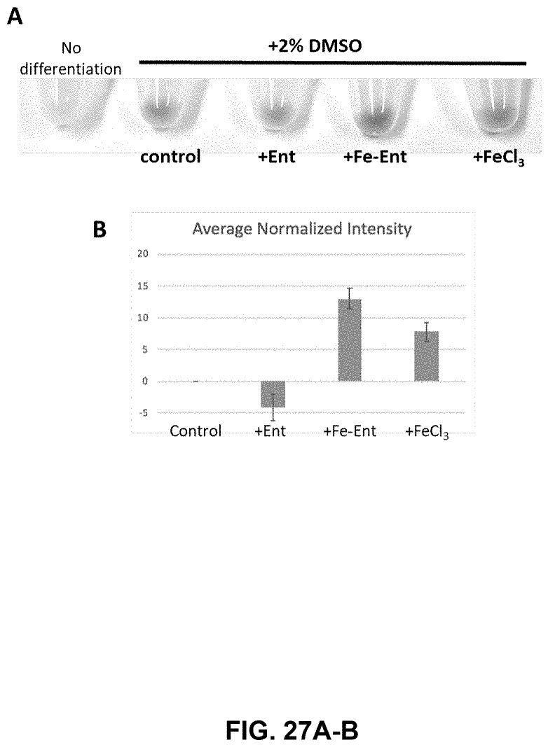

(FIG. 2A). (FIG. 2F) Cartoon diagram and bar graph showing that addition of FeCl₃ to the wild-type E. coli source, which is expected to repress Ent production, inhibited the growth of worms fed heat-killed E. coli. However, this growth was largely recovered by Ent supplementation. Representative worm images are shown in FIG. D. (FIG. 2G) Under an iron-deficient condition with CaEDTA treatment, worms displayed retarded growth. Calcein-AM fluorescence in worms decreases (iron level increase) with the supplementation of either FeCl₃ (in a dosage-dependent manner) or Ent. The effect of Ent supplement on fluorescence level decrease is equivalent to supplementing 10 ul of FeCl₃ (175 ug/ul) to the food. “n”=number of worms scored. Data are represented as mean±SEM. ***P<0.001. All data are representative of at least three independent experiments.

FIG. 3. Bacterial enterobactin binds to the α-subunit of ATP synthase. (FIG. 3A) Schematic diagram of the procedure to identify Ent-binding proteins from whole worm lysates by affinity chromatography using biotin-conjugated Ent. The retained proteins were identified by mass spec analysis. The two proteins identified in two independent experiments are indicated. (FIG. 3B) Cartoon diagram of feeding condition, microscope images and bar graph showing that Ent supplementation failed to rescue growth of animals treated with atp-1(RNAi). ctl-2 RNAi did not alter the benefit of Ent supplementation. (FIG. 3C) An in vivo test for Ent binding to ATP-1. Biotin-Ent was used to pulldown interacting proteins from whole worm lysates, followed by streptavidin-bead purification. Western blot analysis using an anti-ATP5A1 antibody (see FIG. 10A for antibody specificity) to detect ATP-1 in IP. (FIG. 3D-E) In vitro tests for Ent binding to ATP-1. The ATP-1::His tagged protein was bound to biotin-Ent (FIG. 3D) and the binding was increased by increased protein concentration, and decreased by adding excess, non-biotin labeled, Ent (FIG. 3E). (FIG. 3F) Cartoon and bar graph showing that Ent mediates the interaction between Fe³⁺ with ATP-1 in an iron-binding assay. Whole worm lysates were treated with 55FeCl₃ +/− siderophore (Ent, ferrichrome or pyoverdine), followed by immunoprecipitation with anti-ATP5A1. The relative iron level was determined by measuring radioactivity. The presence of Ent resulted in >10-fold increase in 55Fe associated with ATP-1-IP. Data are represented as mean±SEM. ***P<0.001. All data are representative of at least three independent experiments, except D and E (two independent experiments).

FIG. 4. ATP-1, but not the ATP synthase, is required for the Ent role in promoting host iron level. (FIG. 4A-D) Cartoon diagrams of feeding conditions, fluorescence images of calcein-AM staining, and bar graphs of quantitative data depicting the impact of feeding conditions on host iron level. (4A) The host iron level is decreased in atp-1 loss-of-function (lf) homozygous animals (100% L1 arrested, n>50) under regular feeding conditions, and the decrease in iron level, but not growth arrest (100%, n>50), was effectively suppressed by expressing an ATP-binding defective ATP-1 mutant protein from a transgene [Prpl28::atp-1(del)]. Data are represented as mean±SEM. (FIG. 4B) RNAi knockdown of atp-1, but not each of three other subunits of the ATP synthase, caused a decrease in iron level in the worms under regular feeding conditions. Data are represented as mean±SD. (FIG. 4C) Pretreatment of animals with atp-1 RNAi eliminated the benefit of Ent supplementation when animals were fed entF-mutant bacteria, indicating the dependence of the Ent role on ATP-1. Data are represented as mean±SEM. (3D) Pretreatment of animals with atp-1 RNAi eliminated the benefit of Ent supplementation when animals were fed only heat-killed bacteria. Data are represented as mean±SEM. (FIG. 4E). Deletion of 8AA (DRQTGKTA) of the ATP binding domain did not alter the binding of ATP-1 to Ent in the in vitro binding assay similar to that in FIG. 3D. “n”=number of worms scored. ***P<0.001. All data are representative of at least three independent experiments.

FIG. 5. Ent-ATP-1 interaction in mitochondria promotes iron level increase in mitochondria. (FIG. 5A) Cartoon illustration and data from an in vivo mitochondrial iron uptake assay. The worms were fed with 55FeCl₃ +/− Ent. Mitochondria were extracted from these worms and the relative 55Fe level between the two samples for each RNAi treatment was determined. The presence of Ent caused about 3-fold increase in 55Fe level in mitochondria, and the Ent effect was eliminated by RNAi of atp-1, but not by RNAi of other ATP synthase genes. (FIG. 5B) CAS staining assay showing significantly lower mitochondrial siderophore level in worms fed entF-mutant bacteria. (FIG. 5C) atp-1 RNAi caused a reduction in the mitochondrial siderophore level. (FIG. 5D) An in vitro mitochondrial iron uptake assay. Mitochondria were first purified from worm lysates, followed by incubation with 55FeCl₃ +/− Ent and measurement of the relative 55Fe level between the two samples for each RNAi treatment. The presence of Ent led to 10-fold higher 55Fe level in mitochondria and this effect was significantly reduced by RNAi of atp-1, but not by RNAi of other ATP synthase genes. (FIG. 5E and F) Ent supplementation led to increase in the activities of Fe-S cluster-containing enzymes, indicated by the increased activity of mitochondrial aconitase (FIG. 5E) and succinate dehydrogenase (FIG. 5F) in worms fed Ent-deficient food. *P<0.05, **P<0.01, ***P<0.001. Data are represented as mean±SD. All data are representative of at least three independent experiments.

FIG. 6. Ent also promotes mitochondrial iron level in mammalian cells by interacting with the a-subunit of ATP synthase. (FIG. 6A) CAS staining indicating that Ent supplementation led to an increased siderophore level in HEK293T cells. Data are represented as mean±SD. (FIG. 6B) An in vivo Ent-biotin pulldown assay using total protein extracts from human HEK293T cells cultured +/− Biotin-Ent and western blot identified ATP5A1 as an Ent-binding protein. (FIG. 6C) An in vitro test for Ent binding to mammalian ATP5A1. The ATP5A1::His-tagged protein bound to biotin-Ent, and the binding was competed out by excess, non-biotin labeled Ent. (FIG. 6D) Bar graph showing Ent mediates the interaction between ATP5A1 and iron. HEK293T whole cell lysates were treated with 55FeCl₃ +/− Ent, followed by immunoprecipitation with anti-ATP5A1 and measurement of radioactivity. Data are represented as mean±SD. (FIG. 6E) Results of an in vivo mitochondrial iron uptake assay (similar to that in FIG. 5A for C. elegans) showing that Ent supplementation significantly increased Fe³⁺ uptake into mitochondria and the increase was eliminated by siRNA knockdown of ATP5A1. The effectiveness of the siRNA is shown in FIG. 13A. Data are represented as mean±SD. (FIG. 6F) Result of an in vitro mitochondria iron uptake assay showing the ATP5A1-dependent impact of Ent on iron uptake of mitochondria from HEK293T cells. Like in C. elegans (FIG. 5D), addition of Ent boosted iron uptake into mitochondria, and this benefit was sharply reduced by siRNA knock down of ATP5A1. Data are represented as mean±SD. (FIG. 6G) Fluorescence images and quantitative data of HEK293T cells stained with fluorescent mitochondrial iron indicator, RPA. Ent supplementation caused a significant decrease in staining (indicating an increase in iron), which was eliminated by knocking down ATP5A1. Data are represented as mean±SEM. **P<0.01, ***P<0.001. All data are representative of at least three independent experiments.

FIG. 7. Proposed new paradigm for the iron “tug of war” between commensal bacteria and host animals. (FIG. 7A) The discovery of the role of lipocalin 2 (lcn2) led to the classical concept of the iron “tug of war” between pathogenic bacterial and the host immune system. Upon infection, lcn2 is induced to bind to Ent-Fe³⁺, which blocks the role of Ent in acquiring iron from host cells for bacterial growth (Baumler and Sperandio, 2016; Ellermann and Arthur, 2017; Xiao et al., 2017). This sequestering function inhibits bacterial growth but may not benefit host iron homeostasis and other physiological roles. (FIG. 7B) The surprising, beneficial role of Ent-ATP synthase a-subunit in promoting mitochondrial iron concentration points to a new mechanism that was evolved to counteract the known negative effect of Ent on iron homeostasis and thus enhances the symbiotic relationship between gut bacteria and animals.

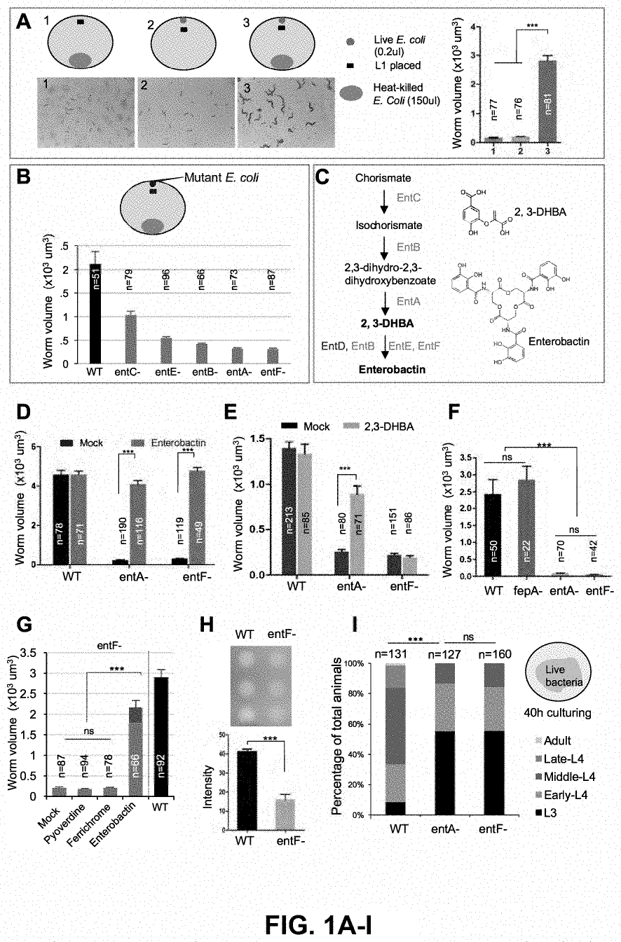

FIG. 8. Bacterial enterobactin promotes C. elegans development. (FIG. 8A) Cartoon diagram of feeding condition, and microscope images showing that worms fed heat-killed E. coli combined with either entA- or entF-mutant E. coli grew slower, and this defect was fully suppressed by Ent supplementation. Quantitative data are shown in FIG. 1D. (FIG. 8B) Cartoon diagram of FepA, the ferric enterobactin receptor on the bacterial outer membrane that facilitates uptake of the Ent-Fe³⁺ complex in E. coli. (FIG. 8C) Cartoon diagram of feeding condition, and microscope images showing that worms fed heat-killed E. coli combined with fepA-mutant E. coli did not show a growth defect, unlike feeding with entA- or entF-mutants. Quantitative data are shown in FIG. 1F. (FIG. 8D) The entA- and entF-mutant E. coli strains exhibited growth rates similar to those of the parental wild-type strain, E. coli K12-BW25113. (8E) The entA- and entF-mutant E. coli strains colonized the host gut as efficiently as the parental wild-type strain. (FIG. 8F) Cartoon diagram of feeding condition, microscope images and bar graph showing that neither pyoverdine nor ferrichrome caused obvious growth defects in worms fed heat-killed food plus wild-type, live E. coli. (FIG. 8G) Fluorescence microscopy of worms containing mtGFP under the same feeding condition as in (FIG. 8F). Supplementation of each of the three siderophores did not affect mitochondrial morphology in the inventive assay system. (FIG. 8H) In the liquid culture, the P. aeruginosa-produced siderophore pyoverdine is toxic to worms as it damages host mitochondria (the mtGFP network pattern is fragmented and reduced to large and punctate bodies) (Kirienko et al., 2015). However, Ent does not disrupt the mitochondrial morphology. (FIG. 81) Cartoon diagram of feeding condition, microscope images, and bar graph showing that Ent supplementation to heat-killed E. coli OP50 did not rescue host development, supporting the idea that multiple bacteria-generated metabolites from live bacteria are needed to support worm growth (Qi et al., 2017). “n” =number of worms scored. Data are represented as mean±SEM. ***P<0.0001.

FIG. 9. Functional relationships between Ent, iron concentration and worm growth. (FIG. 9A) Cartoon illustration of feeding condition, microscope images and bar graph showing that adding more Fe³⁺ (FeCl₃) to heat-killed food did not suppress the growth defect (FIG. 9A) caused by Ent deficiency (see FIG. 1B). Data are represented as mean±SD. (FIG. 9B) Calcein-AM staining showing that, unlike Ent supplementation, adding more Fe³⁺ did not elevate the iron level in worms fed heat-killed E. coli. Data are represented as mean±SEM. (FIG. 9C) Adding more hemin to food did not suppress the growth defect caused by Ent deficiency. Data are represented as mean±SEM. (FIG. 9D) Microscopic images showing that adding more ferric chloride to wild-type E. coli in the novel assay system inhibited worm growth. The growth defect was recovered by Ent supplementation. Quantitative data are shown in FIG. 2F. “n”=number of worms scored.

FIG. 10. In vitro mapping of the Ent-binding sequence of ATP-1. (FIG. 10A) A single band was detected in whole worm extracts by the antibody against mammalian ATP synthase a-subunit, and the band intensity dramatically decreased in atp-1(RNAi)-treated samples, supporting the specificity of this antibody for the worm protein ATP-1. (FIG. 10B-C) In vitro tests for binding between iron-bound Ent and ATP-1. Increasing concentrations of the

ATP 1::His-tagged protein led to increased binding to biotin-Fe-Ent (B). The binding was decreased by adding excess, non-biotin labeled Ent (FIG. 10C). Data are represented as mean±SEM. 10(D) The full length ATP-1 protein sequence was divided into three segments and then expressed in E. coli. The in vitro binding assay using purified proteins showed that the middle segment retains Ent-binding ability. (FIG. 10E) Eight peptides covering the middle segment of ATP-1 (identified in D) were tested for binding, revealing a 21 amino acid peptide (FCIYVAVGQKRSTVAQIVKRL) that was sufficient to bind Ent in the in vitro binding assay. (FIG. 10F) The ATP-1 protein with the 21 amino acid sequence deleted lost the Ent binding ability. Therefore, this 21-residue peptide is both essential and sufficient for Ent binding, even though it may not be sufficient for its iron uptake function.

FIG. 11. ATP-1 binding to Ent is independent of the β-subunit of ATP synthase and sequence comparison between ATP-1 with human ATP5A1. (FIG. 11A) Immunostaining showing that α-subunit co-localizes with β-subunit of ATP synthase in HEK293T cells. (FIG. 11B) atp-1(RNAi) displayed the slow growth phenotype in C. elegans. (11C) Western blot showing that ATP-1 binding to Ent is independent of the β-subunit of ATP synthase (ATP-2). Worms treated with control or atp-2 RNAi were fed Biotin-Ent and total protein extracts were isolated, followed by streptavidin-bead purification. The ATP-1 protein was detected in both samples by Western blot using an antibody against the α-subunit of ATP synthase. Worms grew slower after atp-2(RNA1) treatment, indicating that RNAi was effective in knocking atp-2 down. (FIG. 11D) Protein sequence alignment of the α-subunit of ATP synthases from C. elegans and humans. The predicted ATP and Ent binding sites are indicated.

FIG. 12. ATP-1 co-localizes with MitoTracker. Immunostaining images of dissected intestines showing that ATP-1 co-localizes with MitoTracker. atp-1 RNAi treatment results in reduced immunostaining.

FIG. 13. siRNA effectively reduced the level of ATP5A1. ATP5A1 protein level was decreased in cells treated with siRNA ATP5A1.

FIG. 14. Mice grow slow with entF-bacterial colonization. 5-week old germ-free mice were colonized with wild-type or entF-(enterobactin deficient) bacteria. After colonization, mice grew 4 weeks. Body weight was measured each week and weight increase was calculated.

FIG. 15. 2-D Chemical Structure of enterobactin. (coordinating oxygen atoms are indicated in red).

FIG. 16. Enterobactin and its synthetic analogs: the catecholate TRENCAM and salicylate SERSAM, SER(3M)SAM, TRENSAM and TREN(3M)SAM ligands. (coordinating oxygen atoms are indicated in red).

FIG. 17. Schematic diagram demonstrating exemplary E. coli microorganism in the intestinal lumen secreting the ferric iron-binding compound Ent, taken from G. J. Anderson. “Iron Wars—The Host Strikes Back” The New England Journal of Medicine. Nov. 22, 2018.

FIG. 18. Ent addition increases iron uptake in human HEK293 cells and murine intestinal epithelial (MODE-K) cells in medium with iron chelator. (FIG. 18A) When the iron chelator Deferoxamine (DFO) was added to the medium, the iron level was significantly decreased in HEK293 cells, as indicated by the increase in Calcein AM fluorescence. The iron level was mostly recovered by the addition of Ent (1.5uM) into the medium. The decrease in Calcein AM fluorescence with Ent addition (45%) is stronger than the test without using DFO. (18B) A similar result is seen in murine intestinal epithelial cells (MODE-K) cells tested under the same conditions.

FIG. 19. Ent and ATPSα promote iron traffic across the lipid bilayer of liposomes. (FIG. 19A) Cartoon illustration of experimental conditions and graphic quantification of Calcein AM staining. (FIG. 19B) The Calcein AM dye was added to liposomes +/− ATPSα and the fluorescence intensity of the liposome was measured. (FIG. 19C) Cartoon illustration of experimental conditions and graphic quantification of iron uptake. (FIG. 19D) Liposomes were incubated with radioiabeled Fe³⁺ (⁵⁵FeCl₃) and the radioactivity (relative CPM) of the liposome was measured.

FIG. 20. Ent supplementation by oral gavage led to increase in hemoglobin and spleen iron levels in an anemic mouse model (dietary anemia). 3-week old female mice were fed an iron-deficient diet (IDD) (or control diet) for 6 weeks to induce anemia (confirmed by hemoglobin measurement). Mice (5 per group) were then treated with +/− Ent (two concentrations) or +/− FeSO₄ by oral gavage (once every two days) for two weeks.

FIG. 21. Ent supplemented by drinking water (ad libitum) led to increase in hemoglobin level in an anemic mouse model (dietary anemia). 3-week old male mice were fed an iron-deficient diet (IDD) (or control diet) for 5 weeks to induce anemia. They were then fed IDD +/− Ent added to the drinking water for two more weeks. Fresh dilutions of Ent in water were provided once per week.

FIG. 22. Ent supplemented by drinking water (ad libitum) promotes growth of mice fed control (iron-adequate) diet. 4.5-week male mice were treated with the iron-adequate control diet (CD) used in FIG. 20 and FIG. 21, the matched control for the iron-deficient diet (IDD).

FIG. 23. Ent promotes mouse growth in mice colonized with a single E. coli strain. Supplementing FIG. 14, the inventors demonstrate (FIG. 23A). Five-week old female germ-free (GF) mice were colonized with a single non-pathogenic E. coli (K12) strain, wild type or entF-. Mouse growth (weight gain) was measured for the following 4 weeks. Germ-free (GF) mice colonized with entF-E. coli displayed slower growth compared to mice colonized with wildtype E. coli. Interestingly, the difference in weight gain was greatest in the first two weeks after colonization. (FIG. 23B and C) Iron level of the terminal mice was significantly lower only in the spleen (˜35%) but not in the liver or other tissues, which is consistent with the results seen in FIG. 20. Iron level was measured. (FIG. 23D) Ent supplementation overcomes growth delay in GF mice colonized with entF-E. coli. GF female mice colonized with entF-bacteria were supplemented with Ent [2 concentrations added to the drinking water (pH 5.5) once per week].

FIG. 24. The impact of Enterobactin (Ent) in promoting animal development is not seen with other siderophores. Newly hatched C. elegans larvae were fed wild type K12 E. coli, or entF-E. coli supplemented with the indicated siderophores.

FIG. 25. Ent stability tests of different solvents and pH conditions. (FIG. 25A) Ent stability was measured by the CAS liquid assay (adapted from Arora & Verma 2017). Degradation is indicated by increased absorbance. Ent diluted in H20 (pH 5.5) (with 10% DMSO) degraded rapidly within the first 60 minutes. In contrast, Ent diluted in 100% DMSO showed little change during the assay period. (FIG. 25B) Ent stability in H20 at varying pH was measured by the CAS liquid assay. Ent diluted in H₂O (pH 6.5/7) was more stable than the other dilutions in acidic and basic H₂O. (FIG. 25C) The stability of Ent versus Ent bound to Iron (Fe-Ent or ferric-Ent) was measured by the NGAL fluorescence assay (adapted from Goetz et al., 2002) and graphed with a linear trendline. Ent and Fe-Ent were prepared at the same concentration and in the same buffers. Degradation is indicated by increased relative fluorescence. Fe-Ent was more stable than Ent alone. For all tests, dilutions were kept at room temp and exposed to light. Error is standard deviation of the mean.

FIG. 26. Impact of Ent and Fe-Ent on the growth of iron-deficient C. elegans with a mutation in the smf-3/DMT1 gene. All tests were done on a smf-3/DMT1(-) mutant strain in a culture media where an iron chelator (2,2′-bipyridyl) was added to reduce the environmental iron level (FIG. 26A). Ent supplementation benefits growth of iron-deficient worms and does so by an SMF-3/DMT1 independent mechanism. smf-3(-) mutants display slow growth on test plates. This growth delay was rescued by supplementation with Ent, scored as the percentage of the population at the indicated growth stage. This result suggests the potential of Ent to treat amenia patients with a defect in the DMT1-involved iron uptake system. (FIG. 26B) The Ent benefit is executed through an E. coli-independent mechanism. The entP,fepA: E. coli mutant cannot synthesize or utilize Enterobactin. Supplementation with Ent still rescued the growth of smf-3(-) mutants, even when the E. coli strain could not use Enterobactin. These results suggest that Enterobactin benefits C. elegans by an E. coli-independent mechanism. (FIG. 26C-D) Ferric Ent (Fe-Ent) also benefits worm growth, the benefit is greater than supplementation with Ent or FeCl₃ alone, and the benefit is through an E. coli-independent mechanism. Fe-ENT was made by combining equimolar amounts of purified Enterobactin and FeCl₃ (1:1 binding ratio). Supplementation with Fe-ENT supported growth of smf-3(-) mutants better than supplementation with equimolar Enterobactin or FeCl₃ alone (FIG. 26C) and did so by an E. coli-independent mechanism (D).

FIG. 27. Ferric Enterobactin supplementation benefits differentiation of erythroid progenitor cells (MEL) to red blood cells. (FIG. 27A) MEL cells were grown with the indicated treatments. Cell pellets are shown. (FIG. 27B) Quantification of MEL color change presented as the average intensity of each cell pellet, normalized to the intensity of the control. Error bars are the standard deviation of three technical replicates.

### DETAILED DESCRIPTION OF THE INVENTION

The invention may include novel systems, methods and compositions for the therapeutic administration of Ent, and/or Ent analogs to treat iron-deficiency in a subject. As noted above, enterobactin (Ent), FIG. 15 is a canonical siderophore biosynthesized by Gram-negative species of Enterobacteriaceae that include Escherichia coli (E. coli), Salmonella, and Klebsiella. Decades of exploration pertaining to enterobactin biosynthesis and coordination chemistry, in addition to investigations of the proteins involved in its cellular transport and processing, provide a detailed molecular and physiological understanding of how this chelate contributes to bacterial iron homeostasis and colonization (Raymond et al. Proc. Natl. Acad. Sci. U.S.A 2003, 100, 3584-3588). The enterobactin synthetase is comprised of four proteins, EntBDEF, and is responsible for the production of enterobactin from L-serine and 2,3-dihydroxybenzoic acid (DHB). Following biosynthesis, Ent is exported into the extracellular space where it scavenges Fe³. Enterobactin coordinates Fe³by its three catecholate groups with Ka^{˜}1049 M-1.

For example, one aspect of the present invention relates to methods of treating iron-deficiency, and preferably iron-deficiency anemia in a subject in need thereof, the method including administering to the subject a therapeutically effective amount of Ent, and a pharmaceutically acceptable carrier thereof, and/or a pharmaceutically acceptable salt thereof, and/or a pharmaceutical composition thereof. In this embodiment, Ent may comprise purified, substantially purified and/or isolated Ent which may be further combined with a pharmaceutically acceptable composition, such as an excipient.

Another aspect of the present invention relates to methods of preventing iron-deficiency, and preferably iron-deficiency anemia in a subject in need thereof, the method including administering to the subject a prophylactically effective amount of Ent and/or Ent analog, and a pharmaceutically acceptable carrier thereof, and/or a pharmaceutically acceptable salt thereof, and/or a pharmaceutical composition thereof.

In another example, one aspect of the present invention relates to methods of treating an iron-deficiency in a subject in need thereof, the method including administering to the subject a therapeutically effective amount of an Ent through the introduction of a probiotic and/or symbiotic delivery vector. In another example, one aspect of the present invention relates to methods of treating an iron-deficiency in a subject in need thereof, the method including administering to the subject a prophylactically effective amount of an Ent through the introduction of a probiotic and/or symbiotic delivery vector.

For example, one aspect of the present invention relates to methods of treating iron-deficiency, and preferably iron-deficiency anemia in a subject in need thereof, the method including administering to the subject a therapeutically effective amount of Ent, or an analog thereof through a non-pathogenic symbiotic and/or probiotic bacteria. In this embodiment, the invention may include a genetically modified a non-pathogenic symbiotic and/or probiotic donor bacteria that may be configured to express and/or overexpress Ent in a recipient host. In one embodiment, the genetically modified a non-pathogenic symbiotic and/or probiotic donor bacteria may be configured to express and/or overexpress one or more genes involved in Ent bio-synthesis. For example, in this preferred embodiment, one or more genes involved in Ent bio-synthesis may be part of an expression cassette and further operably linked to an expression control sequence(s). In a preferred embodiment, this promotor may be a constitutive promotor.

In one embodiment, one or more of the above referenced genetically engineered probiotic and/or symbiotic bacteria may be part of a pharmaceutical, and/or nutraceutical composition. In additional embodiments, isolated Ent, and/or one or more of the above referenced genetically engineered probiotic and/or symbiotic bacteria may be part of a food or drink additive that may administer a therapeutically effective amount to treat a disease condition. In another embodiment, isolated Ent, and/or one or more of the above referenced genetically engineered probiotic and/or symbiotic bacteria may be part of a supplement and may further be coupled with an additional dietary supplement, such as a dietary iron supplement.

As used herein, “Enterobactin” or “Ent” is a high affinity siderophore found in microbial systems, and in particular gram-negative bacteria. Ent is a strong siderophore that binds to the ferric ion (Fe3+) with the affinity (K=1052 M-1). As used herein, “Fe-Ent,” “Ent-Fe,” “Ent-Fe³⁺,” or “ferric-Ent” refers to a Enterobactin complexed with iron. An Ent analog may include a Fe-Ent analog, wherein the Ent along is complexed with iron.

A nucleotide or polynucleotide sequence is “operably linked to an expression control sequence(s)” or (e.g., a promoter and, optionally, an enhancer) when the expression control sequence controls and regulates the transcription and/or translation of that polynucleotide sequence. As used herein, the phrase “gene product” refers to an RNA molecule or a protein. Moreover, the term “gene” may sometime refer to the genetic sequence, the transcribed and possibly modified mRNA of that gene, or the translated protein of that mRNA.

Examples of such Ent bio-synthesis genes may include entB, entD, entE, and/or entF. Additional embodiment may include genes that are involved in the biosynthesis of Ent precursors, including entC, entB, and entA. Such genes may be heterologous and or endogenous to the subject and include all homologs and orthologs of the same. It should be noted that the nucleic acid and amino acid sequences of the above referred genes are within the knowledge of those of ordinary skill in the art and are specifically incorporated herein by reference. As used herein, the term “probiotic” generally refers to bacteria that may colonize a target host for sufficient time to deliver a therapeutically effect amount of Ent to said host.

The terms “purified,” “substantially purified,” and “isolated” refer to a compound useful in the present invention being free of other, dissimilar compounds with which the compound is normally associated in its natural state, so that the compound comprises at least 0.5%, 1%, 5%, 10%, 20%, 50%, 75%, 80%, 85%, 90%, 95%, 96%, 97%, 98%, 99%, 99.5%, or 99.9% of the mass, by weight, of a given sample or composition. In one embodiment, these terms refer to the compound comprising at least 95%, 98%, 99%, or 99.9% of the mass, by weight, of a given sample or composition.

The term “pharmaceutically acceptable salt” refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hy droi odi de, 2-hy droxy ethan sul fonate (i sethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable salts include inorganic acids such as hydrochloric acid, hydrobromic acid, sulfuric acid, and phosphoric acid; and organic acids such as oxalic acid, maleic acid, succinic acid, and citric acid. “Basic addition salts” refer to salts derived from appropriate bases, these salts including alkali metal, alkaline earth metal, and quaternary amine salts. Hence, the present invention contemplates sodium, potassium, magnesium, and calcium salts of the compounds disclosed herein, and the like. Basic addition salts can be prepared during the final isolation and purification of the compounds, often by reacting a carboxyl group with a suitable base such as the hydroxide, carbonate, or bicarbonate of a metal cation or with ammonia or an organic primary, secondary, or tertiary amine. The cations of therapeutically acceptable salts include lithium, sodium (by using, e.g., NaOH), potassium (by using, e.g., KOH), calcium (by using, e.g., Ca(OH)₂), magnesium (by using, e.g., Mg(OH)₂ and magnesium acetate), zinc, (by using, e.g., Zn(OH)₂ and zinc acetate), and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine. Other representative organic amines useful for the formation of base addition salts include ethylenediamine, ethanolamine, diethanolamine, piperidine, piperazine, choline hydroxide, hydroxyethyl morpholine, hydroxyethyl pyrrolidone, imidazole, n-methyl-d-glucamine, N,N′-dibenzylethylenediamine, N,N′-di ethyl ethanol amine, N,N′-dimethylethanolamine, triethanolamine, and tromethamine. Basic amino acids (e.g., 1-glycine and 1-arginine) and amino acids which may be zwitterionic at neutral pH (e.g., betaine (N,N,N-trimethylglycine)) are also contemplated.

The terms “administer,” “administering,” or “administration” refers to injecting, implanting, absorbing, ingesting, Ent, which may be part of a pharmaceutical composition, or ingesting a probiotic bacteria configured to produce Ent as described herein, or a probiotic bacteria in a pharmaceutical composition thereof.

The terms “treatment,” “treat,” and “treating” refer to reversing, alleviating, delaying the onset of, or inhibiting the progress of a “pathological condition” (e.g., a disease, disorder, or condition, or one or more signs or symptoms thereof) described herein. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. In a preferred embodiment, treatment may be directed towards an iron-deficiency related disorder, such as iron-deficiency anemia.

A “therapeutically effective amount” of a compound, preferably Ent or an Ent analog, of the present invention or a pharmaceutical composition thereof is an amount sufficient to provide a therapeutic benefit in the treatment of a disease or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, and/or enhances the therapeutic efficacy of another therapeutic agent. A “therapeutically effective amount” may also mean “prophylactically effective amount” of a compound of the present invention is an amount sufficient to prevent a disease or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

Pharmaceutical compositions described herein can be prepared by any method known in the art of pharmacology. In general, such preparatory methods include the steps of bringing the compound Ent or an Ent analog or Ent conjugate, or a probiotic bacteria configured to produce, and or over produce Ent (i.e., the “active ingredient”) into association with a carrier or excipient, and/or one or more other accessory ingredients, and then, if necessary and/or desirable, shaping, and/or packaging the product into a desired single- or multi-dose unit. Pharmaceutical or nutraceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, Poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyani sole, butylated hydroxytoluene, monothioglycerol, potassium metabi sulfite, propionic acid, propyl gallate, sodium ascorbate, sodium bisulfate, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium s orb ate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, German® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, prop anoi c acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs which, preferably contain a unit dosage of Ent, or a unit dosage of a probiotic bacteris configured to express and or over express Ent. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically, contemplated routes of administration of the compounds and compositions disclosed herein are inhalation and intranasal administration, subcutaneous administration, mucosal administration, and interdermal administration. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of an “active ingredient” required to achieve an effective amount will vary from subject to subject, depending, for example, on species, age, and general condition of a subject, severity of the side effects or disorder, identity of the particular compound, mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

Also encompassed by the invention are kits (e.g., pharmaceutical packs). The kits provided may comprise a compound of Ent or composition (e.g., pharmaceutical or diagnostic composition) and a container (e.g., a vial, ampule, bottle, syringe, and/or dispenser package, or other suitable container). The kits provided may comprise antibodies that selectively bind an enterobactin or composition (e.g., pharmaceutical or diagnostic composition) and a container (e.g., a vial, ampule, bottle, syringe, and/or dispenser package, or other suitable container). In some embodiments, provided kits may optionally further include a second container comprising an excipient (e.g., pharmaceutically acceptable excipient) for dilution or suspension of an inventive pharmaceutical composition or compound. In some embodiments, the compound of Ent or composition provided in the first container and the second container are combined to form one unit dosage form. In another aspect, the present invention provides kits including a first container comprising antibodies produced using the compound Ent, i.e., antibodies that selectively bind an enterobactin.

The term “subject” refers to any animal. In certain embodiments, the subject is a mammal. In certain embodiments, the subject is a human (e.g., a man, a woman, or a child). The human may be of either sex and may be at any stage of development. In certain embodiments, the subject has been diagnosed with the condition or disease to be treated. In other embodiments, the subject is at risk of developing the condition or disease. In certain embodiments, the subject is an experimental animal (e.g., mouse, rat, rabbit, dog, pig, or primate). The experimental animal may be genetically engineered. In certain embodiments, the subject is a domesticated animal (e.g., dog, cat, bird, horse, cow, goat, sheep, or chicken).

### EXAMPLES

### Example 1

### A bacterial Mutant Screen Identifies the Benefit of E. coli-Produced Enterobactin to Host Development

In one embodiment of the inventive technology, the present inventors created a unique assay to facilitate the identification of microbial metabolites that benefit growth and development of host animals. Previous studies by the present inventors revealed that heat-killed (HK) E. coli lacks certain molecules that are collectively required for C. elegans larval growth. Larval growth was recovered when the HK E. coli plate was supplemented with a trace amount of live E. coli, which alone could not support worm growth (FIG. 1A), suggesting that the trace amount of live bacteria generated metabolites that rendered HK food usable. This unique feeding condition was used to search for E. coli mutants that fail to support normal worm growth, potentially due to their inability to provide specific metabolites to benefit host animals. After screening an E. coli single-gene knockout library (E. coli Keio collection), the present inventors found that worms fed a trace amount of any of the five E. coli mutants with disrupted enterobactin (Ent) biosynthesis grew significantly slower (FIG. 1B,C). Strikingly, the present inventors observed that worm growth defects were completely overcome by dietary supplementation of Ent (FIG. 1D and FIG. 8A). In addition, supplementing with the metabolic intermediate 2,3-DHBA rescued worm growth on entA- but not entF-E. coli (FIG. 1C, E), confirming that only the final product Ent can provide this benefit to the worm.

The present inventors further showed that this beneficial role of Ent for worm growth is likely independent of the bacterial usage of Ent as a siderophore. Specifically, disrupting fepA, which encodes the E. coli outer membrane receptor for ferric Ent (FIG. 8B), did not affect worm development (FIG. IF and FIG. 8C). The present inventors also found that the entA- and entF-mutant bacteria exhibited no obvious defects in growth under the outlined culture condition or in worm gut colonization (FIG. 8D and E).

Supplementation of two other siderophores (pyoverdine and ferrichrome) failed to generate a similar impact on worm growth (FIG. 1G and FIG. 8F), indicating the specificity of the observed Ent role. A previous study showed that pyoverdine, a siderophore produced by P. aeruginosa, is toxic to C. elegans by damaging the host mitochondria in liquid culture (Kirienko et al., 2015), raising the question of whether the negative results with pyoverdine or ferrichrome were mainly due to the toxicity of these siderophores. The present inventors thus tested and observed, under the identified culturing condition with solid medium, no obvious defects in growth (FIG. 8F) or mitochondrial morphology (FIG. 8G) in C. elegans fed wild-type E. coli and supplemented with pyoverdine or ferrichrome. In addition, the present inventors found that Ent supplementation did not disrupt mitochondrial morphology even in liquid culture (FIG. 8H), which is distinct from the effect of pyoverdine.

An established assay (Schwyn and Neilands, 1987) was modified to evaluate the siderophore level in whole worms, and the present inventor found that worms fed wild-type E. coil contained a significantly higher level of siderophore than worms fed entF-mutant E. coli (FIG. 1H). The production of Ent from E. coli under the culture condition is consistent with a relatively low iron culture media in the referenced experiments, given that high iron level is known to repress Ent biosynthesis (Kwon et al., 1996).

Ent supplementation alone did not result in any appreciable effect on the development of worms fed only HK bacteria (FIG. 81), which indicates that HK bacteria lack more than just Ent or any one specific metabolite (Qi et al., 2017). Conversely, worms fed abundant live entA- or entF-mutant bacteria continued to grow at slower rates (FIG. 11), which confirmed the significant benefit of Ent to host development that was prominently detected by the novel sensitive assay system (FIG. 1BD). Such a benefit of Ent on animal growth may also be pronounced in certain natural environments.

### Example 2

### Bacterial Enterobactin Promotes Iron Pool in the Host

Since Ent has a high affinity for Fe^{3 +}, it may potentially benefit host animals' growth and development by impacting iron homeostasis. The present inventors employed the commonly used fluorescent cell-permeable dye, calcein AM, of which emission is quenched by iron binding, and applied it to live worms as previously described to estimate the overall iron level in the host. Worms fed entA- or entF-mutant bacteria had much lower iron levels, as evidenced by drastically increased fluorescence intensity, and the iron levels were recovered by Ent supplementation (FIG. 2A). The present inventors also examined the expression of the iron responsive gene ftn-2 that encodes a C. elegans homolog of the iron-storage protein ferritin (Romney et al., 2011). The expression of a pftn-2::GFP reporter was dramatically reduced in worms fed entA- or entF-mutant bacteria (FIG. 2B). Therefore, bacterial Ent boosts the iron level in the host C. elegans.

To exclude an effect of worm growth status on the Ent-promoted iron level increase, the present inventors showed, using both iron markers, that worms fed HK E. coli displayed a lower iron level and such a defect was suppressed by Ent supplementation (FIG. 2C,D), while the worms remained arrested under both conditions. Conversely, the iron level was low in worms fed abundant live entA- or entF-mutant bacteria alone (FIG. 2E), where worms continued to grow, albeit at a lower rate (FIG. 1H). Therefore, the Ent impact on the host iron level is independent of other feeding conditions and worm growth. The results from feeding only HK food (FIG. 2C,D) provided additional evidence that the beneficial Ent effect is not due to a secondary effect of bacterial usage of Ent.

The typical worm growth media (NGM plate) seeded with E. coli as food appears to present a low iron environment based on the recipe as well as the fact that Ent biosynthesis in bacteria would be repressed under iron replete conditions. Adding more Fe³⁺ (FeCl₃) to food neither suppressed the growth defect (FIG. 9A) nor raised cellular iron level (FIG. 9B) in animals fed Ent-deficient food. Addition of hemin also did not impact animal growth (FIG. 9C). Therefore, Ent may promote optimal iron uptake and growth of C. elegans regardless of the iron level in food. If Ent is required for optimal C. elegans development, adding more ferric chloride to wild-type E. coli in the invention's novel assay system (FIG. 2F) would be expected to inhibit Ent production in the live E. coli source and consequently slow down worm growth. Indeed, the present inventors observed the worm growth defect with the addition of ferric chloride to live E. coli, and this growth defect was suppressed by Ent supplementation (FIG. 2F, 9D). This supports a requirement of Ent even in iron-rich environment, which may in turn suggest that iron level in the gut of C. elegans does not usually reach the height leading to a full repression of Ent production from the gut microbiota.

Moreover, a previous study showed that under an iron-deficient condition where worms were treated with CaEDTA, the iron level and growth rate were decreased in worms (Kiang et al., 2014) (FIG. 2G). However, both defects were suppressed by either adding more FeCl₃ or Ent supplementation (FIG. 2G). Strikingly, 10× FeCl₃ supplementation recovered the iron level in worms fed CaEDTA to the level observed in worms fed CaEDTA plus additional Ent (FIG. 2G), which indirectly suggests that Ent-mediated iron uptake may be responsible for at least a 10-fold iron level increase in worms under this condition. This result indicates the profound impact of Ent on host iron homeostasis under iron-deficiency condition.

### Example 3

### Bacterial Enterobactin Binds to Host ATP Synthase α-Subunit

To understand the mechanism underlying the Ent effect on worm iron homeostasis, the present inventors employed affinity chromatography using an immobilized Ent and subsequent mass spectrometric analysis to identify Ent-binding proteins in worms (FIG. 3A). Only two candidate proteins, CTL-2 and ATP-1, were captured in both of two independent experiments (FIG. 3A and Table 1). CTL-2 is a homolog of catalases that are known to bind iron. ATP-1 is the a-subunit of the mitochondrial ATP synthase that is not known for a role in iron biology (Junge and Nelson, 2015). The present inventors then tested the requirement of each protein for the Ent effect on animal growth. RNAi of the atp-1 gene, but not ctl-2, prevented the rescue of worm growth by Ent supplementation (FIG. 3B), suggesting that ATP-1, but not CTL-2, could potentially play a critical role in mediating the observed Ent function in the host.

The present inventors then carried out three additional tests to confirm Ent binding to ATP-1. First, in an in vivo assay, worms were fed bacteria +/− Biotin-Ent and total protein extracts were isolated, followed by streptavidin-bead purification and SDS-PAGE. The ATP-1 protein was clearly detected by Western blotting using an antibody against the mammalian α-subunit of ATP synthase (FIG. 3C and FIG. 10A), indicating an interaction between Ent and ATP-1. Second, in an in vitro binding assay, the present inventors found Biotin-Ent (iron free) efficiently bound to ATP-1-His (FIG. 3D), and the binding could be outcompeted by excess Ent (FIG. 3E). Just as the iron-free Biotin-Ent, iron-bound Biotin-Ent also bound the ATP-1 protein (FIG. 10B, C).

Finally, the present inventors tested the ability of Ent to mediate the interaction between ATP-1 and iron. The present inventors added radiolabeled iron (55FeCl₃) to worm lysates and then immunoprecipitated ATP-1. By measuring the radioactivity in the ATP-1-IP sample, the present inventors found that addition of Ent (but not two other siderophores) dramatically increased the binding of ATP-1 to 55Fe (FIG. 3F), supporting a specific role of Ent in mediating the interaction between ATP-1 and iron. Additional analyses indicate that a 21 amino acid sequence of ATP-1 is critically involved in Ent binding (FIG. 10D-F). (Notably, with respect to FIG. 11D, the C. elegans ATP-1 amino acid sequence is identified herein as SEQ ID NO. 7, while human ATP-1 amino acid sequence is identified herein as SEQ ID NO. 8.) Collectively, these results indicate that bacterial Ent directly binds to a eukaryotic ATP-1, which facilitates the ATP-1 interaction with iron.

### Example 4

### ATP-1 is Required for Enterobactin-Dependent Promotion of the Host Iron Level

The present inventors next tested if Ent promotes the host iron pool through the Ent-ATP-1 complex. The present inventors first determined a role of ATP-1 in iron homeostasis by showing that the iron level was dramatically decreased in an ATP-1 loss-of-function (if) C. elegans mutant, or wild-type worms treated with 43.-1(RNA1), as indicated by the increase in the calcein-AM fluorescence (FIG. 4A, B). The present inventors then determined that the Ent effect in promoting iron level in the worm was dependent on ATP-1, as RNAi of atp-1 eliminated the iron level gain seen with Ent supplementation to entF-mutant bacteria (FIG. 4C). The HK E. coli +/− Ent effect on growth-arrested animals (FIG. 2C) was also found to be dependent on atp-1 (FIG. 4D). Finally, the host iron level was not significantly changed by RNAi knockdown of each of three other subunits of the ATP synthase (FIG. 4B), suggesting that the ATP-1 impact on iron level was unlikely due to an indirect effect of disrupting the ATP synthase function. Therefore, bacterial Ent promotes host iron homeostasis through its interaction with the ATP synthase a-subunit.

### Example 5

### The observed ATP-1 Function is Independent of its Role in the ATP Synthase

As the ATP synthase a-subunit, ATP-1 is expected to interact with β and other subunits of this large enzyme complex. Using immunostaining, the present inventors observed co-localization of the a-subunit (ATP5A1) and β-subunit (ATP5B) of the mammalian ATP synthase in mitochondria (FIG. 11A). Consistent with the essential role of the ATP synthase, loss-of-function mutations in both atp-1 and atp-2 display L1 arrest phenotypes in C. elegans (FIG. 4A). RNAi of 4)-1 or atp-2 also displayed growth defects (FIG. 11B,C). We thus tested if the ATP-1 function in promoting the host iron pool is dependent on other subunits of the ATP synthase. We found that ATP-1 binding to Ent is not affected by RNAi of the β-subunit of ATP synthase (ATP-2) (FIG. 11C) and as indicated in FIG. 4B, the decrease of iron level caused by atp-1(RNAi) was not seen in worms treated with RNAi of genes for the β, b and O subunits. Therefore, this role of ATP-1 is independent of other ATP synthase subunits.

Since binding to ATP is a critical part of the role the α-subunit in ATP synthase, the present inventors sought to determine if the ATP binding domain is required for the Ent interaction and the role in promoting iron level. As such, the present inventors deleted residues 198-205 (DRQTGKTA) from the ATP-1 protein sequence (FIG. 11D) and found, by the in vitro binding assay, that this deletion did not reduce the ability of this protein to bind to Ent (FIG. 4E), suggesting that ATP-1-Ent binding is independent of ATP-1-ATP binding. The present inventors next tested if transgenic expression of this ATP-1(del) protein was sufficient to function as ATP-1 in Ent-mediated iron uptake. As indicated in FIG. 4A, expression of this protein behind a ribosome gene promoter from the [Prp1-28::atp-1(del)] transgene significantly suppressed the iron level decrease caused by the atp-1(lf) mutation. Therefore, the ATP-1 function, both in interacting with Ent and promoting iron uptake, is independent of its role in ATP binding.

### Example 6

### Enterobactin-ATP-1 Interaction Promotes Iron Level in Host Mitochondria

Since iron transport into mitochondria critically affects the labile iron pool and overall iron homeostasis, the present inventors sought to determine if bacterial Ent and its interaction with host ATP-1 promotes iron level in mitochondria. The present inventors first observed colocalization of ATP-1 with the MitoTracker marker in the intestine (FIG. 12A), which is consistent with ATP-1 function in mitochondria. The present inventors then carried out an in vivo assay, modified from a published protocol for mammalian cells (Devireddy et al., 2010), to examine the role of the Ent-ATP-1 interaction in promoting mitochondrial iron level. Worms were treated with RNAi and fed 55FeCl₃ +/− Ent, followed by isolation of mitochondria and measurement of radioactivity (55Fe). Mitochondrial iron (55Fe) was increased by three-fold with Ent supplementation, and this increase depended on ATP-1, but not other ATP synthase subunits (FIG. 5A). In addition, the present inventors showed that mitochondria isolated from worms fed wild-type E. coli contained a significantly higher level of siderophore than worms fed entF-mutant bacteria, indicating that Ent also enters mitochondria (FIG. 5B), and the level of Ent in mitochondria was significantly reduced when ATP-1 was reduced by RNAi (FIG. 5C). Therefore, bacterial Ent facilitates host mitochondrial iron level increase and this process requires a novel, ATP synthase-independent function of ATP-1 in host mitochondria.

### Example 7

### ATP-1 Acts in Mitochondria to Facilitate Ent-Fe3 +/− Level Increase in Mitochondria

The ATP synthase a-subunit resides in the mitochondrial matrix, and this protein is transported into mitochondria by a well-characterized mitochondrial protein transport mechanism. Therefore, it is possible that ATP-1 facilitates Ent-Fe³⁺ import into mitochondria by a “co-transport” model, which requires ATP-1 binding to Ent prior to the transport. To test this model, the present inventors sought to determine if it was possible to observe the roles of Ent and ATP-1 in purified mitochondria, where there is no ATP-1 synthesis or shuttling to mitochondria.

In this in vitro assay modified from a published protocol for mammalian cells (Devireddy et al., 2010; incorporated herein by reference), the present inventors first extracted mitochondria from RNAi treated worms and then added 55FeCl₃ +/− Ent, followed by quantification of 55Fe. Addition of Ent dramatically increased iron (55Fe) level in mitochondria by 10-fold and this increase was largely reduced when the worms were treated with RNAi of atp-1, but not for worms treated with RNAi targeting the three other ATP synthase subunits (FIG. 5D). This result further supports the role of Ent and ATP-1, but not the rest of the ATP synthase, in promoting mitochondrial iron level. Since no new ATP-1 protein could be made in the assay mix, the results of this assay likely exclude the potential “cotransport” model and may suggest that ATP-1 facilitates mitochondrial Ent-Fe import by binding to Ent within mitochondria. This “retention” model in turn suggests that Ent-Fe³⁺ enters mitochondria by other means and may have a high tendency to exit without the ATP-1 interaction, which may be consistent with a hypothesis that Ent can enter mammalian cells by passive permeation. The observed stronger Ent effect in the in vitro assay (FIG. 5D), compared to the in vivo assay (FIG. 5A), may be due to a stronger affinity of Ent for the mitochondrial environment over the solution under the in vitro assay condition.

To observe the functional impact of the Ent-mediated increase in mitochondrial iron level, the present inventors tested the effect of Ent on iron-dependent mitochondrial enzymes in worms under the present inventor's culturing condition. It was found that activities of aconitase and succinate dehydrogenase, two mitochondrial Fe-S cluster enzymes, were significantly increased by Ent supplementation (FIG. 5E, F), supporting the role of the Ent-ATP-1 complex in supplying iron to Fe-S clusters and other iron-containing molecules in mitochondria.

### Example 8

### Ent Interacts with ATP5A1 to Promote Mitochondrial Iron Uptake into Mammalian Cells

Sequence alignment indicates 78% identity between the ATP synthase a-subunits from C. elegans (ATP-1, Wormbase; SEQ ID NO. 7) and humans (ATP5A1, NCBI; SEQ ID NO. 8) (FIG. 11D). Using CAS staining, the present inventors observed that Ent supplemented to the culture medium can enter human HEK293T cells (FIG. 6A). To test if this Ent-ATP-1 function is conserved in mammals, the present inventors repeated the prior described biotin-Ent pulldown assay (FIG. 3A) using total protein extracts from human HEK293T cells cultured +/− Biotin-Ent. ATP5A1 was clearly detected (FIG. 6B), supporting that Ent also binds to ATP5A1 in mammalian cells. In an in vitro binding assay, biotin-Ent directly bound to the human protein ATP5A1 and the binding was effectively competed away by the presence of excess, free Ent (FIG. 6C). In a third assay, the present inventors added radiolabeled iron (55FeCl₃) to the cell lysates +/− Ent, followed by IP using the ATP5A1 antibody. The presence of Ent increased the level of 55Fe in the ATP5A1-IP samples (FIG. 6D). Together, these data indicate that the interaction between Ent and the ATP synthase a-subunit is conserved in mammalian cells.

To test if the function of the Ent-ATP-1 complex in promoting mitochondrial iron uptake is also conserved in human cells, the present inventors performed both an in vivo and an in vitro mitochondrial iron uptake assay and found that addition of Ent prominently increased iron level in mitochondria in both assays (FIG. 6E, F). Moreover, using siRNA knockdown (FIG. 13A), the present inventors observed that this Ent-mediated increase depended on ATP5A1 (FIG. 6E, F) in a similar manner as that in the assays for C. elegans (FIG. 5A, D). The present inventors also measured the mitochondrial iron level in cells by using the fluorescent mitochondrial iron indicator, RPA, to which iron binding quenches its fluorescence. Supplementation with Ent increased the mitochondrial iron level in the cells, and this iron-boosting effect was not seen in cells treated with ATP5A1 siRNAi (FIG. 6G). The relatively smaller changes seen for cultured cells, compared to the difference in worms fed heat-killed food (FIG. 2), may potentially be due to the higher base iron level under the cell culturing condition. These data support that Ent impacts the mammalian mitochondrial iron level and does so in an ATP5A1-dependent manner.

### Example 9

### Ent Addition Increases Iron Uptake in Human HEK293 cells in Medium with Iron Chelator

In FIG. 2G, the inventors showed the strong effect of Ent to move iron into live C. elegans under iron-deficient conditions (iron chelator added). Here, a similar test was done in human HEK293 cells. When the iron chelator Deferoxamine (DFO) was added to the medium, the iron level was significantly decreased in cells, as indicated by the increase in Calcein AM staining fluorescence. The iron level was mostly recovered by the addition of Ent (1.5 uM) into the medium. The decrease in Calcein AM fluorescence with Ent addition (45%) is stronger than the test without using DFO shown in FIG. 6G, suggesting that the low iron condition is more sensitive to the presence of Ent. This result demonstrates the effectiveness of Ent in moving iron, either the low-level free iron or iron bound to DFO, into cells.

### Example 10

### Ent and ATPSα Promote Iron Traffic Across the Lipid Bilayer of Liposomes

The inventor has used synthetic liposomes to test the ability of Ent to move iron across the lipid bilayer in vitro. Briefly, FeCl₃ +/− Ent (1.5 uM) were added to fresh liposomes formed from commercial lipids (Avanti Lipids). ATPSα (ATP-1 or ATP5A1) was added to the liposome by an established method. As shown in FIG. 19, the level of Fe³⁺ associated with the liposome was measured by two methods: (A) Calcein N_M staining. The Calcein AM dye was added to liposomes +/− ATPSα and the fluorescence intensity of the liposome was measured; (B) Liposomes were incubated with radiolabel ed Fe³⁺ (''FeCl₃) and the radioactivity (relative CPM) of the liposome was measured. Method (A) measures iron inside the liposome, since calcein AM is only inside. Method (B) is a simpler method that may not exclude iron on the outside of the liposome. in each test, the presence of Ent clearly boosted the level of iron either inside (A) or associated with (B) the liposome.

### Example 11

### Ent Supplementation by Oral Gavage Led to Increase in Hemoglobin and Spleen Iron Levels in an Anemic Mouse Model (Dietary Anemia)

The inventors fed 3-week old female mice an iron-deficient diet (IDD) (or control diet) for 6 weeks to induce anemia (confirmed by hemoglobin measurement). Mice (5 per group) were then treated with +/− Ent (two concentrations) or +/− FeSO₄ by oral gavage (once every two days) for two weeks. As generally shown in FIGS. 20A-B, feeding IDD caused a drastic reduction of both hemoglobin and iron levels. Ent supplementation partially but significantly recovered the hemoglobin level, albeit with large error bars, suggesting a likely role of Ent in increasing iron uptake efficiency under a severe anemic condition. The iron level was increased in the spleen, but not in the liver, with Ent addition. A commercial kit (BioAssay system) was used to obtain hemoglobin level. Under normal conditions, ˜70% of the iron absorbed from the intestine is used to generate hemoglobin, and only a small percent may be stored in the liver or other somatic tissues if iron is in excess. Under anemic conditions, a great percentage of iron that has entered mitochondria (then bound to heme) may be funneled to erythropoiesis. Therefore, it was not unexpected to see little impact of Ent supplementation on liver iron level (iron storage).

### Example 12

### Ent Supplemented by Drinking Water (Ad Libitum) Led to Increase in Hemoglobin Level in an Anemic Mouse Model (Dietary Anemia)

The inventors fed 3-week old male mice an iron-deficient diet (IDD) (or control diet) for 5 weeks to induce anemia. They were then fed IDD +/− Ent added to the drinking water for two more weeks. Fresh dilutions of Ent in water were provided once per week. As generally shown in FIG. 21, mice continuously fed the iron-adequate control diet (CD) were included as the control. Hemoglobin level was measured by a Hemavet Blood Analyzer. Mean value±SD is shown. Ent supplementation significantly improved the hemoglobin level in the anemic mice. Because Ent was later found to be highly unstable in the deionized water used in the test (pH 5.5) (see FIG. 25), the effect of Ent was likely reduced in this test. Future tests will administer Ent in pH-adjusted water and will include more frequent fresh dilutions of Ent to ensure stability.

### Example 13

### Ent Supplemented by Drinking Water (Ad Libitum) Promotes Growth of Mice Fed Control (Iron-Adequate) Diet

The present inventors treated 4.5-week male mice with the iron-adequate control diet (CD) described above, the matched control for the iron-deficient diet (IDD). As shown above in FIGS. 20 and 21, mice fed this diet had relative normal hemoglobin and iron levels. As further shown in FIG. 22, when supplemented with Ent in drinking water (pH 5.5), the mice had an increased growth rate. This experiment was incomplete because the hemoglobin and iron levels were not measured, which will be repeated. However, the weight gain data is still meaningful because the inventors had already shown that Ent has a profound role on larval growth in C. elegans and that effect was due to the ability to promote iron level increase in the animals (FIGS. 1 and 2).

### Example 14

### Ent Promotes Mouse Growth Mice Colonized with a Single E. coli Strain

As generally shown in FIG. 23, (A) Five-week old female germ-free (GF) mice were colonized with a single non-pathogenic E. coli (K12) strain, wild type or entF-(Ent-deficient E. coli). Mouse growth (weight gain) was measured for the following 4 weeks. Germ-free (GF) mice colonized with entF-E. coli displayed slower growth compared to mice colonized with wildtype E. coli. Interestingly, the difference in weight gain was greatest in the first two weeks after colonization. (B and C) Iron level of the terminal mice was significantly lower only in the spleen (˜35%) but not in the liver or other tissues, which is consistent with the results seen in FIG. 20. Iron level was measured. (D) Ent supplementation overcomes growth delay in GF mice colonized with entF(-) E. coli. GF female mice colonized with entF-bacteria were supplemented with Ent [2 concentrations added to the drinking water (pH 5.5) once per week].

The data indicate a significant recovery of growth. The Ent effect was more obvious in the first 2 weeks. It may be noted, because of the instability of Ent in water with low pH, the Ent effect here may be limited. Additional embodiments may administer Ent in pH-adjusted water, and further include more frequent fresh dilutions of Ent to ensure stability. Since Ent is used by E. coli to support their growth, there may be decreased colonization in the gut without Ent, and the developmental delay in the first 2 weeks could be due to an indirect effect of less E. coli in the gut. However, such a large weight is unlikely due to the potential difference in E. coli colonization. More importantly, the inventors have already shown that the role of Ent in promoting C. elegans development is independent of bacterial usage of Ent (see FIG. 1F) and Ent biosynthesis does not have an obvious effect on gut colonization in C. elegans (Qi and Han, 2018). The difference in weight gain between weeks 1-2 and weeks 3-4 may suggest a more prominent role of Ent (or E. coli) in earlier stages, where the rapid growth requires more iron acquisition. Alternatively, mice might have some adaptive/compensatory changes in the latter two weeks (less dependent on Ent).

### Example 15

### The Impact of Enterobactin (Ent) in Promoting Animal Development is not seen with other Siderophores

Following the inventors' established assay (Qi and Han, 2018), as shown in FIG. 24, newly hatched C. elegans larvae were fed wild type K12 E. coli, or entF-E. coli supplemented with the indicated siderophores. In this assay, Ent is necessary to support C. elegans growth. The volume of the worms was measured 3 days later (as was done for the experiments described in FIG. 1). While Enterobactin supplementation recovers the growth defect seen with entF-, the other five siderophores tested fail to show an effect (consistent with other results in FIG. 1G). Therefore, the role of Ent in promoting iron transport and animal development is specific to Ent.

### Example 16

### Ent Addition Increases Iron Uptake in Human HEK293 Cells and Murine Intestinal Epithelial (MODE-K) Cells in Medium with Iron Chelator

In human HEK293 cells with the standard culture medium, Ent addition promoted a significant but modest increase of cellular iron uptake (˜33%, data not shown). The present inventors then created a low-iron condition by adding an iron chelator, Deferoxamine (DFO) to the medium (FIG. 18A). The iron level was largely recovered by the addition of Ent (-50%), supporting the beneficial role of Ent in promoting iron uptake. The assay was also performed using mouse intestinal epithelial (MODE-K) cells. While adding Ent alone modestly increased the cellular iron level (decrease in fluorescence) in cells in standard medium, the effect was more pronounced with the iron-poor/DFO condition (FIG. 18B).

### Example 17

### Impact of Ent and Fe-Ent on the Growth of Iron-Deficient C. elegans with a Mutation in the smf-3/DMT1 Gene

SMF-3/DMT1 is a conserved divalent metal transporter that transports iron into intestinal cells. The smf-3/DMT1 mutant has been established as an iron deficiency model in C. elegans. These mutants have defective iron uptake that results in iron deficiency (Romney et al 2011) and delayed growth under iron-poor growth conditions created by addition of an iron chelator (2,2′-bipyridyl) to the culture media (Raj an et al 2019). The present inventors employed this model to test the impact of Enterobactin (Ent) on C. elegans growth under iron deficiency and found that ENT supplementation rescued the growth of smf-3(-) mutants to adulthood, compared to control (FIG. 26A). This result suggests that Ent benefits C. elegans growth under iron-poor/iron-deficient conditions and does so by an SMF-3/DMT1-independent mechanism. In addition, this data also suggest that Ent may be an effective treatment for human anemia patients with deficiency in the DMT1-involved iron uptake system.

To determine if the Enterobactin supplementation was benefiting the worm directly or benefiting the worm through an E. coli-dependent mechanism, the test was repeated using an E. coli double mutant that cannot synthesize or utilize Ent (entP,fepk). Again, Ent supplementation rescued the growth of smf-3(-) mutants to adulthood, compared to control (FIG. 26B). This result suggests that Ent benefits the worm through an E. coli-independent mechanism.

In addition to supplementation with Enterobactin (iron-free), the test was also performed with Ferric Enterobactin (iron-bound Enterobactin, Fe-ENT) or an equimolar amount of FeCl₃ alone. smf-3/DMT1 growth was rescued with Fe-Ent faster and to a greater extent than observed for Enterobactin or FeCl₃ supplementation alone (FIG. 26C). This benefit is also independent of E. coli (entP,fepA: test, right panel, FIG. 26D). These results suggest that Ferric Enterobactin is bioavailable to C. elegans and this form may be the most stable and effective form to support growth in this iron deficiency model.

### Example 18

### Ferric Enterobactin (Fe-Ent) Promotes Erythroid Differentiation in Murine Erythroid Precursor Cells

MEL cells are murine erythroid progenitor cells that are arrested at the proerythroblast stage. In the laboratory, these cells are induced to undergo erythroid differentiation to red blood cells by addition of various chemicals (e.g. 2% DMSO) (Friend, 1971). Erythroid differentiation is an iron-dependent process that results in a color change in the differentiated cells. The present inventors tested if addition of Fe-Ent would positively impact the differentiation of wild-type MEL cells to red blood cells. We found that addition of Fe-Ent resulted in an increase in MEL differentiation, and this increase was better than supplementation with equimolar FeCl₃ alone (FIG. 27). Supplementing with equimolar free Ent had the opposite effect and showed a decrease in differentiation. These data suggest that Fe-Ent benefits the iron-dependent differentiation of murine erythroid progenitor cells.

### Example 19

### Material and Methods

C. elegans strains and maintenance. Nematode stocks were maintained on nematode growth medium (NGM) plates seeded with bacteria (E. coli OP50) at 20° C. The following strains/alleles were obtained from the Caenorhabditis Genetics Center (CGC): N2 Bristol (termed wild type), VC2824: H28016.1(ok2203) FhT2 [bli-4(e937) let-?(q782) qIs48] (I;III). XA6901: qaEx6901 [ftn-2p::pes-10::GFP::his +lin-15(+)],5J4103: zcIs14 [myo-3::GFP(mit)]. For Prp1-28:atp-1(del) transgene, the full coding region deleted ATP binding sequence (residues 198-205: DRQTGKTA) was cloned into pPD95.77, driven by a ubiquitous RPL28 promoter, then lOng/u1 plasmid with 5ng/ul injection marker (pCFJ90) was injected in atp-1 (1P mutant (VC2824).

Cell line. HEK293T cells were obtained from ATCC and were maintained in a humidified cabinet at 37° C. with 5% CO2. Cells were cultured with DMEM supplemented with 10% FBS, 4 mM L-Glutamine, 100 units penicillin per mL, 100 ₁.tg streptomycin per mL, and 0.25 μg amphotericin B per mL.

E. coli Keio collection screen. Preparation of heat-killed (HK) OP50 plates followed the procedure described previously (Qi et al., 2017). Standard overnight culture of E. coli OP50 grown in LB broth was concentrated to 1/10 vol and was then heat-killed in a 75° C. water bath for 90 min. The 150 pi of the HK-OP50 was spread onto one side of NGM plate. For preparation of bacterial mutant assay plates, E. coli Keio (Baba et al., 2006) mutants were grown overnight at 37° C. in LB medium with 10 mg/mL kanamycin. 0.2 uL of the bacterial culture (OD₆₀₀) were seeded to the other side of HK OP50 plate. About 300 synchronized L1 worms were added to the screen plate, and cultured at 20° C., then scored for worm size at day 3 and day 4. The entire library was used for the primary screen; each bacterial mutant was screened once. For the secondary screen, 200 candidate mutants were screened (3 replicates) to confirm the slow growth phenotype.

Chemical supplementation of culture plates. For chemical supplementation, each chemical was dissolved in water or DMSO to generate a stock. The stock solution was added to HK OP50 and then spotted onto NGM plates. The chemical name, vendor, stock concentrations and volumes used for each chemical are listed as follows: 2,3-DHBA (Sigma 126209-5G, 300mM, 5 μl), Enterobactin (Sigma E3910-1MG, 1 mg/ml, 5 μl), Pyoverdine (Sigma P8124-1MG, 0.5 mg/ml, 5 μl), Ferrichrome (Sigma F8014-1MG, 0.5mg/ml, 5 μl), Hermin (Sigma 51280, 1 mg/ml, 5 μl), FeCl₃ (Sigma 236489, 175 ug/ul, volumes indicated in assay). For CaEDTA supplementation (Kiang et al., 2014), 50 ul of CaEDTA (50 ug/ul) was spread onto the center of the NGM plates seeded with OP50, then different volume of the FeCl3[175 ug/ul] (0 ul, 1 ul, 5ul, 10 ul, 50 l) or 20 ul of the Ent (0.5mg/ml) was added onto the center of the bacterial lawn. Bacterial growth analyses. Overnight cultures were diluted to a final OD₆₀₀=0.01 in 200 μL of NGM liquid medium. Bacteria were grown in 96-well plates with shaking in a Synergy2 plate reader (BioTek) at 37° C. for 17 h. The OD₆₀₀ was recorded at 20 min intervals.

Bacterial colonization in worm assays. Bacterial colonization of C. elegans was determined using a method adapted from a published procedure (Portal-Celhay and Blaser, 2012). Briefly, L3 staged worms were collected from NGM plates, and extensively rinsed with 10mL M9 buffer 3 times. The animals were then put on empty NGM plates with 100 mg/mL ampicillin for 1 hr to remove surface bacteria. 10 worms were individually picked into M9 buffer and homogenized by sonication. Part or all of the mixture was then plated onto LB plates. After incubation at 37 ° C. overnight, the number of bacterial colonies were determined.

Quantification of siderophores. CAS agar plates were prepared according to the published method (Schwyn and Neilands, 1987). Worms were feed wild-type or entF-mutant E. coli then collected, washed 5 times with 10mL M9, then the worms were starved in 10mL M9 overnight to digest and clear intestinal bacteria. The worms were then homogenized by sonication and the protein concentration of the supernatant was measured by BCA Protein Assay Kit (ThermoFisher, 23225). Protein input was normalized based on protein concentration. Supernatants were placed on CAS agar plates and incubated overnight at room temperature and monitored for orange-colored halo formation and color intensity was quantified by Image J. CAS gives a distinctive blue color when in complex with iron. When the iron is chelated by siderophores, orange halos develop around the sample. The intensity of halo formation is directly proportional to the concentration of siderophores.

Analysis of larval growth by measuring worm size. Synchronized L1 worms were seeded on the indicated NGM plate and grew to the indicated times. Photos were taken and worms volume in each photo were measured by WormSizer software (Moore et al., 2013).

Iron determination in worms. Live imaging of iron in worms was done as previously described (James et al., 2015). Briefly, worms were collected at different culture conditions, then co-cultured in M9 with 0.05 ug/ML calcein-AM (Invitrogen) for 1 h, then washed 3 times in 1 ml M9. Samples were then mounted for fluorescence microscopy.

Western blot. To measure the level of ATP synthase a-subunit, worms treated with RNAi or cells treated with siRNA were analyzed by standard Western blot methods and probed with anti-ATP synthase α-subunit (dilution=1:5000; ThermoFisher,43-9800) and anti-Actin (dilution=1:5000; Sigma-A2066) as a loading control.

Isolation of Enterobactin-binding proteins by biotin-IP and LC-MS. Total proteins were extracted from mixed stage worms and then pre-cleaned three times by adding 100 ul Dynabeads® M-280 Streptavidin. Equal volumes of these total protein extracts were then separated to two tubes. Biotin-Ent (5 ug) was added into one tube for IP and Biotin alone (5 ug) was added to another tube as the control, both were incubated overnight at 4° C. After incubating with Dynabeads® M-280 Streptavidin for 2 hours, the beads were washed at least 3 times by 1mL PBS. PBS was then removed from the beads and 200 uL 0.1 M ammonium bicarbonate (ABC)/0.001% deoxycholic acid (DCA) was added. The samples were reduced using 5 mM (final) TCEP at 60° C. for 30 min and alkylated using 15 mM iodoacetamide at room temperature for 20 min. 0.5 ug of trypsin was added to each sample and incubated overnight. The samples were then acidified using 7 uL of formic acid. DCA was removed from the samples by phasetransfer using ethyl acetate. The samples were desalted using a Pierce C18 spin column, and dried using a speed vac. The samples were reconstituted in 10 uL Buffer A (0.1% formic acid in water), of which 5 uL was subjected to LC-MSMS analysis.

Enterobactin and ATP-1 protein interaction assays. In vivo binding assay: Worms were allowed to grow with Ent-biotin (5 ug/ml) dietary supplementation, followed by streptavidin-bead IP. Western blot was performed to detect ATP-1, using an antibody against the mammalian ATP synthase a-subunit (Thermo Fisher 43-9800).

In vitro binding assay. (a) Ent-biotin pull-down of total proteins: Ent-biotin and streptavidin beads were used to pull down interacting proteins from worm total protein extracts (same method as that in initial screen for Ent-binding proteins). Western blots were performed to detect ATP-1 (Thermo Fisher 43-9800). (b) Binding of Ent-biotin to purified ATP-1 ::HIS-tagged protein: Purified proteins were treated with Ent-biotin (1 ug/uL; 1 mg/ml stock in DMSO) +/− Ent (1 ug/ul) in assay buffer (50 mM 2-[4-(2-hydroxyethyl)-1-piperazinyl] ethanesulfonic acid (HEPES), pH 8.0, 100 mM NaCl, 0.5 mM dithiothreitol (DTT)) at 30° C. for 1 h, total assay volume was 20 uL. The assay was then quenched with a standard 5×SDS-PAGE_loading buffer (reducing). Proteins were separated by SDS-PAGE and transferred to_nitrocellulose membranes. The membranes were blocked for 1 h with 5% BSA in Trisbuffered saline (TBS) with 0.1% Tween 20 (TBST) at room temperature, followed by incubation for 1 h with horseradish peroxidase streptavidin (Cell Signaling_Technology,3999S) in TBST. After four washes with changes every 15 min in TBST, the_biotinylated proteins were visualized by enhanced chemiluminescence (GE Healthcare,_RPN2232). Ka was calculated as the concentration of ATP-1-His protein when binding_to Ent was at ½ of the maximal level. (c) Dependence of Ent for ATP-1 to interact with iron: Radiolabeled iron (55FeCl₃, 1 uCi) or 55FeCl₃ (1 uCi)+Ent (2 ug) were added to wormlysates and then immunoprecipitated using the antibody against ATP-1 (dilution=1:5000; ThermoFisher,43 9800). After IP, the amount of 55Fe was determined by liquid scintillation.

Immunofluorescence. Antibody staining was performed as previously described (Zhang et al., 2007). Briefly, L1 wild-type worms (N2) were treated by feeding 4)-1 RNAi and grew to L4 stage. Worms were grown for 1 day at 20° C. on NGM agar supplemented with 1 μg/ml MitoTracker Red (Cell Signaling #9082) before antibody staining. Dissected worms were fixed in 3% formaldehyde with 6 mM K2HPO4 (pH 7.2) and 75% methanol for 10 min at −20° C. The fixed worms were rinsed three times in PBS and blocked in PBS containing 0.5% BSA and 0.1% Tween-20 for 1 hr at room temperature. The anti-ATP synthase a-subunit (diluted at 1:200) and Anti-Rabbit antibody (diluted at 1:400) (Invitrogen, A11011), were used as primary and secondary antibodies, respectively.

RNAi treatment. L1 worms were treated by feeding RNAi (Ahringer, Reverse genetics, WormBook 2006) for the first generation and grew to adult. They were then bleached and allowed to hatch in M9 buffer for 18hr. The synchronized L1 worms were seeded on the heat-killed OP50 plate with entF-bacteria or heat-killed OP50 plate supplemented with Ent. After 4 days, worm size was measured. To assay the role of different ATP synthase subunits on iron level in worms, L1 worms were treated with feeding RNAi and grew to young adult. Iron level was measured for worms at the same stage. For in vitro/vivo mitochondrial iron uptake, L1 worms were treated with RNAi targeting the indicated ATP synthase subunits and grew to young adult before being subjected to further procedures.

siRNA treatment in mammalian cells. ATP5A1 siRNA was purchased from Sigma (SASI_Hs01_00119735). Lipofectamine® RNAiMAX Transfection Reagent (ThermoFisher, 13778075) was used for delivery of siRNA into the HEK293T cells, following the manufacturer's instructions. Knockdown efficiency was assessed by immunoblotting.

Mitochondrial iron uptake assays. For the in vitro mitochondrial iron uptake assay, the present inventors modified a published procedure for analysis of mammalian cells (Devireddy et al., 2010). Specifically, 1 uCi 55FeCl₃ was incubated with 2 ug iron-free Ent (1 mg/ml in DMSO) or DMSO at room temperature for 3 hours, followed by the addition of purified mitochondria from worms treated with different RNAi, or cells treated with siRNA. The samples were incubated for 4 hours at room temperature, and the amount of 55Fe in lysed mitochondria was determined by liquid scintillation.

The in vivo mitochondrial iron uptake assay was also modified based a published procedure (Devireddy et al., 2010). Specifically, 1 uCi 55FeCl₃ was incubated with 2 ug iron-free Ent (1 mg/ml stock in DMSO) or DMSO at room temperature 3 hours. 55FeCl₃+DMSO or 55FeCl₃+Enterobactin were added to young adult worms treated with RNAi for first generation. After the worms grew overnight, they were washed in M9. Mitochondria were then isolated followed by measuring the amount of incorporated 55Fe by liquid scintillation.

Mitochondrial iron measurement in mammalian cells. The mitochondrial iron pools were determined as described (Mena et al., 2015). Briefly, cells were loaded for 20 min at 37 ° C. with 2 uM of the mitochondrial iron chelator rhodamine B-[(1,10-phenanthrolin-5-yl) aminocarbonyl]benzyl ester (RPA). After washing, the cells were imaged by fluorescence microscopy.

Mitochondria extraction. Mitochondria Isolation Kit for Cultured Cells (ThermoFisher,89874) was used to extract mitochondria from HEK293T cells. Mitochondria Isolation Kit for Tissue (ThermoFisher, 89801) was used to extract mitochondria from worms.

Enzymatic activity. Succinate Dehydrogenase (MAK197; Sigma) and aconitase (MAK051; Sigma) enzymatic activity were measured using the kits according to the manufacturer's protocol. Briefly, L1 worms were seed on the assay plate +/− Ent. After 48 hours culturing, worms were lysed in ice-cold conditions using the lysis buffer provided in the kit, supplemented with protease. Equal amounts of protein were used for the enzymatic activity assay.

Microscopy. Analysis of fluorescence was performed under Nomarski optics on a Zeiss Axioplan2 microscope with a Zeiss AxioCam MRm CCD camera. Plate phenotypes were observed using a Leica MZ16F dissecting microscope with a Hamamatsu C4742-95 CCD camera.

Quantification. ImageJ software was used for quantifying calcein-AM staining and western blots for Ent-protein binding assay. For calcein-AM staining, the original images taken with GFP channel were used to measure the intensity. The value of staining intensity was determined by subtracting the background intensity from the calcein-AM stained intestine.

Statistical analysis. All statistical analyses were performed using Student's t-test and p<0.05 was considered a significant difference, except FIG. 1I, 2G and 11B which were analyzed by using the x two test.

### Tables

TABLE 1
Potential Ent binding Proteins identified by mass spectrometry analysis.

| | | Gene | Peptides | Peptides | Mol. weight |
|---|---|---|---|---|---|
| | Protein names | names | control | IP | [kDa] |
| 1st IP | Myosin-3 | myo-3 | 0 | 46 | 225.51 |
| | Myosin-4 | unc-54 | 0 | 17 | 224.75 |
| | Myosin-1 | let-75 | 0 | 4 | 223.32 |
| | Myosin-2 | myo-2 | 0 | 9 | 223.05 |
| | Membrane-associated protein gex-3 | gex-3 | 0 | 2 | 129.92 |
| | Paramyosin | unc-15 | 0 | 3 | 101.95 |
| | Peroxisomal catalase 1 | ctl-2 | 0 | 7 | 57.466 |
| | ATP synthase subunit alpha, | atp-1 | 0 | 3 | 55.02 |
| | mitochondrial | act-5 | 0 | 6 | 41.872 |
| | Protein unc-87 | unc-87 | 0 | 4 | 41.576 |
| | Myosin regulatory light chain 2 and | mlc-2; mlc-1 | 0 | 3 | 18.603 |
| | chain 1 | | | | |
| | Myosin, essential light chain | mlc-3 | 0 | 3 | 17.144 |
| | | K08D12.3 | 0 | 2 | 15.557 |
| 2nd IP | Peroxisomal catalase 1 | ctl-2 | 0 | 4 | 57.466 |
| | ATP synthase subunit alpha, | atp-1 | 0 | 2 | 55.02 |
| | mitochondrial | fln-1 | 0 | 1 | 225.1 |
| | | tac-1 | 0 | 1 | 28.543 |
| | | mtd-1 | 0 | 1 | 31.379 |
| | Elongation factor 2 | eef-2 | 0 | 1 | 93.406 |
| | Transitional endoplasmic reticulum | cdc-48.2 | 0 | 1 | 89.639 |
| | ATPase homolog 2 | T21B10.3 | 0 | 1 | 135.53 |
| | Mitogen-activated protein kinase kinase | gck-2 | 0 | 1 | 92.377 |
| | kinase kinase | Y22D7AR.7 | 0 | 1 | 89.024 |
| | | csp-1 | 0 | 1 | 16.921 |

### REFERENCES

The following references are hereby incorporated in their entirety by reference:

[1] Baba, T., Ara, T., Hasegawa, M., Takai, Y., Okumura, Y., Baba, M., Datsenko, K. A., Tomita, M., Wanner, B. L., and Mori, H. (2006). Construction of Escherichia coli K-12 in-frame, single-gene knockout mutants: the Keio collection. Mol Syst Biol 2, 2006 0008.

[2] Barratt, M. J., Lebrilla, C., Shapiro, H. Y., and Gordon, J. I. (2017). The Gut Microbiota, Food Science, and Human Nutrition: A Timely Marriage. Cell Host Microbe 22, 134-141. Baumler, A. J., and Sperandio, V. (2016). Interactions between the microbiota and pathogenic bacteria in the gut. Nature 535, 85-93.

[3] Berg, M., Stenuit, B., Ho, J., Wang, A., Parke, C., Knight, M., Alvarez-Cohen, L., and Shapira, M. (2016). Assembly of the Caenorhabditis elegans gut microbiota from diverse soil microbial environments. ISME J 10, 1998-2009.

[4] Blanton, L. V., Barratt, M. J., Charbonneau, M. R., Ahmed, T., and Gordon, J. I. (2016). Childhood undernutrition, the gut microbiota, and microbiota-directed therapeutics. Science 352, 1533.

[5] Brown, J. M., and Hazen, S. L. (2018). Microbial modulation of cardiovascular disease. Nat Rev Microbiol 16, 171-181.

[6] Cabreiro, F., Au, C., Leung, K. Y., Vergara-Irigaray, N., Cocheme, H. M., Noori, T., Weinkove, D., Schuster, E., Greene, N. D., and Gems, D. (2013). Metformin retards aging in C. elegans by altering microbial folate and methionine metabolism. Cell 153, 228-239.

[7] Cassat, J. E., and Skaar, E. P. (2013). Iron in infection and immunity. Cell Host Microbe 13, 509-519.

[8] Charbonneau, M. R., O'Donnell, D., Blanton, L. V., Totten, S. M., Davis, J. C., Barratt, M. J., Cheng, J., Guruge, J., Talcott, M., Bain, J. R., et al. (2016). Sialylated Milk Oligosaccharides Promote Microbiota-Dependent Growth in Models of Infant Undernutrition. Cell 164, 859-871.

[9] Chelikani, P., Fita, I., and Loewen, P. C. (2004). Diversity of structures and properties among catalases. Cell Mol Life Sci 61, 192-208. Devireddy, L. R., Gazin, C., Zhu, X., and Green, M. R. (2005). A cell-surface receptor for lipocalin 24p3 selectively mediates apoptosis and iron uptake. Cell 123, 1293-1305.

[10] Devireddy, L. R., Hart, D. O., Goetz, D. H., and Green, M. R. (2010). A mammalian siderophore synthesized by an enzyme with a bacterial homolog involved in enterobactin production. Cell 141, 1006-1017.

[11] Dirksen, P., Marsh, S. A., Braker, I., Heitland, N., Wagner, S., Nakad, R., Mader, S., Petersen, C., Kowallik, V., Rosenstiel, P., et al. (2016). The native microbiome of the nematode Caenorhabditis elegans: gateway to a new host-microbiome model. BMC Biol 14, 38.

[12] Donia, M. S., and Fischbach, M. A. (2015). HUMAN MICROBIOTA. Small molecules from the human microbiota. Science 349, 1254766.

[13] Ellermann, M., and Arthur, J. C. (2017). Siderophore-mediated iron acquisition and modulation of host-bacterial interactions. Free Radic Biol Med 105, 68-78.

[14] Flo, T. H., Smith, K. D., Sato, S., Rodriguez, D. J., Holmes, M. A., Strong, R. K., Akira, S., and Aderem, A. (2004). Lipocalin 2 mediates an innate immune response to bacterial infection by sequestrating iron. Nature 432, 917-921.

[15] Ford, S. A., Kao, D., Williams, D., and King, K. C. (2016). Microbe-mediated host defense drives the evolution of reduced pathogen virulence. Nat Commun 7, 13430.

[16] Froissard, M., Belgareh-Touze, N., Dias, M., Buisson, N., Camadro, J. M., Haguenauer-Tsapis, R., and Lesuisse, E. (2007). Trafficking of siderophore transporters in Saccharomyces cerevisiae and intracellular fate of ferrioxamine B conjugates. Traffic 8, 1601-1616.

[17] Garcia-Gonzalez, A. P., Ritter, A. D., Shrestha, S., Andersen, E. C., Yilmaz, L. S., and Walhout, A. J. M. (2017). Bacterial Metabolism Affects the C. elegans Response to Cancer Chemotherapeutics. Cell 169, 431-441 e438.

[18] Grillo, A. S., SantaMaria, A. M., Kafina, M. D., Cioffi, A. G., Huston, N. C., Han, M., Seo, Y. A., Yien, Y. Y., Nardone, C., Menon, A. V., et al. (2017). Restored iron transport by a small molecule promotes absorption and hemoglobinization in animals. Science 356, 608-616.

[19] Gusarov, I., Gautier, L., Smolentseva, O., Shamovsky, I., Eremina, S., Mironov, A., and Nudler, E. (2013). Bacterial nitric oxide extends the lifespan of C. elegans. Cell 152, 818-830.

[20] Han, B., Sivaramakrishnan, P., Lin, C.J., Neve, I. A. A., He, J., Tay, L. W. R., Sowa, J. N., Sizovs, A., Du, G., Wang, J., et al. (2017). Microbial Genetic Composition Tunes Host Longevity. Cell 169, 1249-1262 e1213.

[21] Hider, R.C., and Kong, X. (2010). Chemistry and biology of siderophores. Nat Prod Rep 27, 637-657.

[22] James, S. A., Roberts, B. R., Hare, D. J., de Jonge, M. D., Birchall, I. E., Jenkins, N. L., Cherny, R. A., Bush, A. I., and McColl, G. (2015). Direct in vivo imaging of ferrous iron dyshomeostasis in ageing Caenorhabditis elegans. Chem Sci 6, 2952-2962.

[23] Johnson, D. C., Dean, D. R., Smith, A. D., and Johnson, M. K. (2005). Structure, function, and formation of biological iron-sulfur clusters. Annu Rev Biochem 74, 247-281.

[14] Junge, W., and Nelson, N. (2015). ATP synthase. Annu Rev Biochem 84, 631-657. Kakhlon, O., and Cabantchik, Z. I. (2002). The labile iron pool: characterization, measurement, and participation in cellular processes(1). Free Radic Biol Med 33, 1037-1046.

[15] Kirienko, N. V., Ausubel, F. M., and Ruvkun, G. (2015). Mitophagy confers resistance to siderophore-mediated killing by Pseudomonas aeruginosa. Proc Natl Acad Sci U S A 112, 1821-1826.

[16] Kirienko, N. V., Kirienko, D. R., Larkins-Ford, J., Wahlby, C., Ruvkun, G., and Ausubel, F. M. (2013). Pseudomonas aeruginosa disrupts Caenorhabditis elegans iron homeostasis, causing a hypoxic response and death. Cell Host Microbe 13, 406-416.

[17] Klang, I. M., Schilling, B., Sorensen, D. J., Sahu, A. K., Kapahi, P., Andersen, J. K., Swoboda, P., Killilea, D. W., Gibson, B. W., and Lithgow, G. J. (2014). Iron promotes protein insolubility and aging in C. elegans. Aging (Albany N.Y.) 6, 975-991.

[18] Koppel, N., and Balskus, E. P. (2016). Exploring and Understanding the Biochemical Diversity of the Human Microbiota. Cell Chem Biol 23, 18-30.

[19] Kortman, G. A., Dutilh, B. E., Maathuis, A. J., Engelke, U. F., Boekhorst, J., Keegan, K. P., Nielsen, F. G., Betley, J., Weir, J. C., Kingsbury, Z., et al. (2015). Microbial Metabolism Shifts Towards anAdverse Profile with Supplementary Iron in the TIM-2 In vitro Model of the Human Colon. Front Microbiol 6, 1481.

[20] Kundu, P., Blacher, E., Elinav, E., and Pettersson, S. (2017). Our Gut Microbiome: The Evolving Inner Self. Cell 171, 1481-1493.

[21] Kwon, O., Hudspeth, M. E., and Meganathan, R. (1996). Anaerobic biosynthesis of enterobactin Escherichia coli: regulation of entC gene expression and evidence against its involvement in menaquinone (vitamin K2) biosynthesis. J Bacteriol 178, 3252-3259.

[22] Leulier, F., MacNeil, L. T., Lee, W. J., Rawls, J. F., Cani, P. D., Schwarzer, M., Zhao, L., and Simpson, S. J. (2017). Integrative Physiology: At the Crossroads of Nutrition, Microbiota, Animal Physiology, and Human Health. Cell Metab 25, 522-534.

[23] Liu, J., Rutz, J. M., Feix, J. B., and Klebba, P. E. (1993). Permeability properties of a large gated channel within the ferric enterobactin receptor, FepA. Proc Natl Acad Sci U S A 90, 10653-10657.

[24] Lloyd-Price, J., Mahurkar, A., Rahnavard, G., Crabtree, J., Orvis, J., Hall, A. B., Brady, A., Creasy, H. H., McCracken, C., Giglio, M. G., et al. (2017). Strains, functions and dynamics in the expanded Human Microbiome Project. Nature 550, 61-66.

[25] Meisel, J. D., Panda, O., Mahanti, P., Schroeder, F. C., and Kim, D. H. (2014). Chemosensation of bacterial secondary metabolites modulates neuroendocrine signaling and behavior of C. elegans. Cell 159, 267-280.

[26] Mena, N. P., Garcia-Beltran, O., Lourido, F., Urrutia, P. J., Mena, R., Castro-Castillo, V., Cassels, B. K., and Nunez, M. T. (2015). The novel mitochondrial iron chelator 5-((methylamino)methyl)-8-hydroxyquinoline protects against mitochondrial-induced oxidative damage and neuronal death. Biochem Biophys Res Commun 463, 787-792.

[27] Moore, B. T., Jordan, J. M., and Baugh, L. R. (2013). WormSizer: high-throughput analysis of nematode size and shape. PLoS One 8, e57142.

[28] Muckenthaler, M. U., Rivella, S., Hentze, M. W., and Galy, B. (2017). A Red Carpet for Iron Metabolism. Cell 168, 344-361.

[29] Portal-Celhay, C., and Blaser, M. J. (2012). Competition and resilience between founder and introduced bacteria in the Caenorhabditis elegans gut. Infect Immun 80, 1288-1299.

[30] Postler, T. S., and Ghosh, S. (2017). Understanding the Holobiont: How Microbial Metabolites Affect Human Health and Shape the Immune System. Cell Metab 26, 110-130.

[31] Qi, B., Kniazeva, M., and Han, M. (2017). A vitamin-B2-sensing mechanism that regulates gut protease activity to impact animal's food behavior and growth. Elife 6.

[32] Rangan, K. J., Pedicord, V. A., Wang, Y. C., Kim, B., Lu, Y., Shaham, S., Mucida, D., and Hang, H. C. (2016). A secreted bacterial peptidoglycan hydrolase enhances tolerance to enteric pathogens. Science 353, 1434-1437.

[33] Rauen, U., Springer, A., Weisheit, D., Petrat, F., Korth, H. G., de Groot, H., and Sustmann, R. (2007). Assessment of chelatable mitochondrial iron by using mitochondrion-selective fluorescent iron indicators with different iron-binding affinities. Chembiochem 8, 341-352.

[34] Raymond, K. N., Dertz, E. A., and Kim, S. S. (2003). Enterobactin: an archetype for microbial iron transport. Proc Natl Acad Sci U S A 100, 3584-3588.

[35] Romney, S. J., Newman, B. S., Thacker, C., and Leibold, E. A. (2011). HIF-1 regulates iron homeostasis in Caenorhabditis elegans by activation and inhibition of genes involved in iron uptake and storage. PLoS Genet 7, e1002394.

[36] Saha, P., Yeoh, B. S., Olvera, R. A., Xiao, X., Singh, V., Awasthi, D., Subramanian, B. C., Chen, Q., Dikshit, M., Wang, Y., et al. (2017). Bacterial Siderophores Hijack Neutrophil Functions. J Immunol 198, 4293-4303.

[37] Samuel, B. S., Rowedder, H., Braendle, C., Felix, M. A., and Ruvkun, G. (2016). Caenorhabditis elegans responses to bacteria from its natural habitats. Proc Natl Acad Sci U S A 113, E3941-3949.

[38] Schwyn, B., and Neilands, J. B. (1987). Universal chemical assay for the detection and determination of siderophores. Anal Biochem 160, 47-56.

[40] Scott, T.A., Quintaneiro, L. M., Norvaisas, P., Lui, P. P., Wilson, M. P., Leung, K. Y., Herrera-Dominguez, L., Sudiwala, S., Pessia, A., Clayton, P. T., et al. (2017). Host-Microbe Cometabolism Dictates Cancer Drug Efficacy in C. elegans. Cell 169, 442-456 e418.

[41] Searle, L. J., Meric, G., Porcelli, I., Sheppard, S. K., and Lucchini, S. (2015). Variation in siderophore biosynthetic gene distribution and production across environmental and faecal populations of Escherichia coli. PLoS One 10, e0117906.

[41] Stiernagle, T. (2006). Maintenance of C. elegans. WormBook, 1-11. Tenaillon, O., Skurnik, D., Picard, B., and Denamur, E. (2010). The population genetics of commensal Escherichia coli. Nat Rev Microbiol 8, 207-217.

[42] Tsang, W. Y., and Lemire, B. D. (2003). Mitochondrial ATP synthase controls larval development cell nonautonomously in Caenorhabditis elegans. Dev Dyn 226, 719-726.

[43] Vuong, H. E., Yano, J. M., Fung, T. C., and Hsiao, E. Y. (2017). The Microbiome and Host Behavior. Annu Rev Neurosci 40, 21-49. WHO (2002). Iron deficiency anemia: assessment, preventiion, and control; a guide for programmer managers. Geneva. s.

[44] Wiedemann, N., and Pfanner, N. (2017). Mitochondrial Machineries for Protein Import and Assembly. Annu Rev Biochem 86, 685-714.

[45] Wilson, B. R., Bogdan, A. R., Miyazawa, M., Hashimoto, K., and Tsuji, Y. (2016). Siderophores in Iron Metabolism: From Mechanism to Therapy Potential. Trends Mol Med 22, 1077-1090.

[46] Xiao, X., Yeoh, B. S., and Vijay-Kumar, M. (2017). Lipocalin 2: An Emerging Player in Iron Homeostasis and Inflammation. Annu Rev Nutr 37, 103-130.

[47] Yu, D. J., Hu, J., Huang, Y., Shen, H. B., Qi, Y., Tang, Z. M., and Yang, J. Y. (2013). TargetATPsite: a template-free method for ATP-binding sites prediction with residue evolution image sparse representation and classifier ensemble. J Comput Chem 34, 974-985.

[48] Zhang, L., Ding, L., Cheung, T. H., Dong, M. Q., Chen, J., Sewell, A. K., Liu, X., Yates, J. R., 3rd, and Han, M. (2007). Systematic identification of C. elegans miRISC proteins, miRNAs, and mRNA targets by their interactions with GW182 proteins AIN-1 and AIN-2. Mol Cell 28, 598-613.

[49] Zheng, T., Bullock, J. L., and Nolan, E. M. (2012). Siderophore-mediated cargo delivery to the cytoplasm of Escherichia coli and Pseudomonas aeruginosa: syntheses of monofunctionalized enterobactin scaffolds and evaluation of enterobactin-cargo conjugate uptake. J Am Chem Soc 134, 18388-18400.

[50] Abergel, Rebecca J. et al. “Enterobactin Protonation and Iron Release: Structural Characterization of the Salicylate Coordination Shift in Ferric Enterobactin.” Journal of the American Chemical Society 128.27 (2006): 8920-8931. PMC. Web. 17 July 2018.

[51] Anderson, G.J. (2018). Iron Wars—The Host Strikes Back. N Engl J Med 379, 2078-2080.

[52] Arora N K and Verma M. (2017). Modified microplate method for rapid and efficient estimation of siderophore produced by bacteria. 3 Biotech. 7: 381.

[53] Auerbach, M., and Macdougall, I. (2017). The available intravenous iron formulations: History, efficacy, and toxicology. Hemodial Int 21 Suppl 1, S83-S92.

[54] Brissot, P., Bardou-Jacquet, E., Jouanolle, A. M., and Loreal, O. (2011). Iron disorders of genetic origin: a changing world. Trends Mol Med 17, 707-713.

[55] Cook, J. D., and Reddy, M. B. (1995). Efficacy of weekly compared with daily iron supplementation. Am J Clin Nutr 62, 117-120.

[56] Goetz D. H., Holmes M. A., Borregaard, N., Bluhm M. E., Raymond K. N., Strong R. K. (2002) The neutrophil lipocalin NGAL is a bacteriostatic agent that interferes with siderophore-mediated iron acquisition. Mol Cell. 2002. 10:1033-43.

[57] Jaeggi, T., Kortman, G. A., Moretti, D., Chassard, C., Holding, P., Dostal, A., Boekhorst, J., Timmerman, H. M., Swinkels, D. W., Tjalsma, H., et al. (2015). Iron fortification adversely affects the gut microbiome, increases pathogen abundance and induces intestinal inflammation in Kenyan infants. Gut 64, 731-742.

[58] Kortman, G. A., Dutilh, B. E., Maathuis, A. J., Engelke, U. F., Boekhorst, J., Keegan, K. P., Nielsen, F. G., Betley, J., Weir, J. C., Kingsbury, Z., et al. (2015). Microbial Metabolism Shifts Towards an Adverse Profile with Supplementary Iron in the TIM-2 In vitro Model of the Human Colon. Front Microbiol 6, 1481.

[59] Koutroubakis, I. E., Ramos-Rivers, C., Regueiro, M., Koutroumpakis, E., Click, B., Schoen, R. E., Hashash, J. G., Schwartz, M., Swoger, J., Baidoo, L., et al. (2015). Persistent or Recurrent Anemia Is Associated With Severe and Disabling Inflammatory Bowel Disease. Clin Gastroenterol Hepatol 13, 1760-1766.

[60] Lund, E. K., Wharf, S. G., Fairweather-Tait, S. J., and Johnson, I. T. (1999). Oral ferrous sulfate supplements increase the free radical-generating capacity of feces from healthy volunteers. Am J Clin Nutr 69, 250-255.

[61] Moretti, D., Goede, J. S., Zeder, C., Jiskra, M., Chatzinakou, V., Tjalsma, H., Melse-Boonstra, A., Brittenham, G., Swinkels, D. W., and Zimmermann, M. B. (2015). Oral iron supplements increase hepcidin and decrease iron absorption from daily or twice-daily doses in iron-depleted young women. Blood 126, 1981-1989.

[62] Muckenthaler, M. U., Rivella, S., Hentze, M. W., and Galy, B. (2017). A Red Carpet for Iron Metabolism. Cell 168, 344-361.

[63] Munoz, M., Gomez-Ramirez, S., and Garcia-Erce, J. A. (2009). Intravenous iron in inflammatory bowel disease. World J Gastroenterol 15, 4666-4674.

[64] Qi, B., and Han, M. (2018). Microbial Siderophore Enterobactin Promotes Mitochondrial Iron Uptake and Development of the Host via Interaction with ATP Synthase. Cell 175, 571-582 e511.

[65] Riemer, J., Hoepken, H. H., Czerwinska, H., Robinson, S. R., and Dringen, R. (2004). Colorimetric ferrozine-based assay for the quantitation of iron in cultured cells. Anal Biochem 331, 370-375.

[66] Sazawal, S., Black, R. E., Ramsan, M., Chwaya, H. M., Stoltzfus, R. J., Dutta, A., Dhingra, U., Kabole, I., Deb, S., Othman, M. K., et al. (2006). Effects of routine prophylactic supplementation with iron and folic acid on admission to hospital and mortality in preschool children in a high malaria transmission setting: community-based, randomised, placebo-controlled trial. Lancet 367, 133-143.

[67] Shen, C., Rathore, S. S., Yu, H., Gulbranson, D. R., Hua, R., Zhang, C., Schoppa, N. E., and Shen, J. (2015). The trans-SNARE-regulating function of Munc18-1 is essential to synaptic exocytosis. Nat Commun 6, 8852.

[68] Stevens, G. A., Finucane, M. M., De-Regil, L. M., Paciorek, C. J., Flaxman, S. R., Branca, F., Pena-Rosas, J. P., Bhutta, Z. A., Ezzati, M., and Nutrition Impact Model Study, G. (2013). Global, regional, and national trends in haemoglobin concentration and prevalence of total and severe anaemia in children and pregnant and non-pregnant women for 1995-2011: a systematic analysis of population-representative data. Lancet Glob Health 1, e16-25.

[69] Tang, M., Frank, D. N., Hendricks, A. E., Ir, D., Esamai, F., Liechty, E., Hambidge, K. M., and Krebs, N. F. (2017). Iron in Micronutrient Powder Promotes an Unfavorable Gut Microbiota in Kenyan Infants. Nutrients 9.

[70] WHO (2015). The Global Prevalence of anaemia.

[71] Yu, H., Rathore, S. S., Shen, C., Liu, Y., Ouyang, Y., Stowell, M. H., and Shen, J. (2015). Reconstituting Intracellular Vesicle Fusion Reactions: The Essential Role of Macromolecular Crowding. J Am Chem Soc 137, 12873-12883.

[72] Zhang, A. S., and Enns, C. A. (2009). Iron Homeostasis: Recently Identified Proteins Provide Insight into Novel Control Mechanisms. Journal of Biological Chemistry 284, 711-715.

[73] Rajan M, Anderson C P, Rindler P M, Romney S J, Ferreira Dos Santos M C, Gertz J, Leibold E A. “NHR-14 loss of function couples intestinal iron uptake with innate immunity in C. elegans through PQM-1 signaling” ELife . 2019 Sep 18;8:e44674.

[74] Romney S J, Newman B S, Thacker C, Leibold E A. “HIF-1 regulates iron homeostasis in Caenorhabditis elegans by activation and inhibition of genes involved in iron uptake and storage” PLoS Genetics. 2011 Dec;7(12):e1002394.

[75] C. Friend, W. Scher, J. G. Holland, T. Sato, Proc. Natl. Acad. Sci. U.S.A. 68, 378-382 (1971),

SEQUENCE IDENTIFICATION

SEQ ID NO. 1
EntB
Amino Acid
E. Coli
MAIPKLQAYALPESHDIPQNKVDWAFEPQRAALLIHDMQDYFVSFWGENCPMMEQVIANIAALRDYCKQH
NIPVYYTAQPKEQSDEDRALLNDMWGPGLTRSPEQQKVVDRLTPDADDTVLVKWRYSAFHRSPLEQMLKE
SGRNQLIITGVYAHIGCMTTATDAFMRDIKPFMVADALADFSRDEHLMSLKYVAGRSGRVVMTEELLPAP
VPASKAALREVILPLLDESDEPFDDDNLIDYGLDSVRMMALAARWRKVHGDIDFVMLAKNPTIDAWWKLL
SREVK
SEQ ID NO. 2
EntD
Amino Acid
E. Coli
MRHHRTVLPLAGYTIQQIDFDPATFQPEDLFWLPYHASLTGWGRKRQAEHLAGRIAAAYALREVGEKRLP
AIGDQRQPLWPTPWFGSISHCGQRALAVIADRPVGVDIERRFTPQLAAELESSITSPAEKTALLRSGLPF
PLALTLAFSAKESGFKACHPDVQAGVGENDFTLAAIKEGNLRLRLSTVEYRLQWIQAGEYIITLCAP
SEQ ID NO. 3
EntE
Amino Acid
E. Coli
MSIPFTRWPEEFARRYREKGYWQDLPLTDILTRHAASDSIAVIDGERQLSYRELNQAADNLACSLRRQGI
KPGETALVQLGNVAELYITFFALLKLGVAPVLALFSHQRSELNAYASQIEPALLIADRQHALFSGDDFLN
TFVTEHSSIRVVQLHNDSGEHNLQDAINHPAEDFTATPSPADEVAYFQLSGGTTGTPKLIPRTHNDYYYS
VRRSVEICQFTQQTRYLCAIPAAHNYAMSSPGSLGVFLAGGTVVLAADPSATLCFPLIEKHQVNVTALVP
PAVSLWLQALTEGESRAQLASLKLLQVGGARLSATLAARIPAEIGCQLQQVFGMAEGLVNYTRLDDSAEK
IIHTQGYPMCPDDEVWVADAEGNPLPQGEVGRLMTRGPYTFRGYYKSPQHNASAFDANGFYCSGDLISID
PEGYITVQGREKDQINRGGEKIAAEEIENLLLRHPAVIYAALVSMEDELMGEKSCAYLVVKEPLRAVQVR
RFLREQGIAEFKLPDRVECVDSLPLTAVGKVDKKQLRQWLASRASA
SEQ ID NO. 4
EntF
Amino Acid
E. Coli
MAATMRLTGRLGHCVSAAVTGVLPAVAGSPLAYSDTDEFYPVAGGTMSQHLPLVAAQPGIWMAEKLSELP
SAWSVAHYVELTGEVDSPLLARAVVAGLAQADTLRMRFTEDNGEVWQWVDDALTFELPEIIDLRTNIDPH
GTAQALMQADLQQDLRVDSGKPLVFHQLIQVADNRWYWYQRYHHLLVDGFSFPAITRQTANIYCTWLRGE
PTPASPFTPFADVVEEYQQYRESEAWQRDAAFWAEQRRQLPPPASLSPAPLPGRSASADILRLKLEFTDG
EFRQLATQLSGVQRTDLALALAALWLGRLCNRMDYAAGFIFMRRLGSAALTATGPVLNVLPLGIHIAAQE
TLPELATRLAAQLKKMRRHQRYDAEQIVRDSGRAAGDEPLFGPVLNIKVFDYQLDIPDVQAQTHTLATGP
VNDLELALFPDVHGDLSIEILANKQRYDEPTLIQHAERLKMLIAQFAADPALLCGDVDIMLPGEYAQLAQ
INATQVEIPETTLSALVAEQAAKTPDAPALADARYLFSYREMREQVVALANLLRERGVKPGDSVAVALPR
SVFLTLALHAIVEAGAAWLPLDTGYPDDRLKMMLEDARPSLLITTDDQLPRFSDVPNLTSLCYNAPLTPQ
GSAPLQLSQPHHTAYIIFTSGSTGRPKGVMVGQTAIVNRLLWMQNHYPLTGEDVVAQKTPCSFDVSVWEF
FWPFIAGAKLVMAEPEAHRDPLAMQQFFAEYGVTTTHFVPSMLAAFVASLTPQTARQNCATLKQVFCSGE
ALPADLCREWQQLTGAPLHNLYGPTEAAVDVSWYPAFGEELAQVRGSSVPIGYPVWNTGLRILDAMMHPV
PPGVAGDLYLTGIQLAQGYLGRPDLTASRFIADPFAPGERMYRTGDVARWLDNGAVEYLGRSDDQLKIRG
QRIELGEIDRVMQALPDVEQAVTHACVINQAAATGGDARQLVGYLVSQSGLPLDTSALQAQLRETLPPHM
VPVVLLQLPQLPLSANGKLDRKALPLPELKTQASGRAPKAGSETIIAAAFASLLGCDVQDADADFFALGG
HSLLAMKLAAQLSRQFARQVTPGQVMVASTVAKLATIIDGEEDSSRRMGFETILPLREGNGPTLFCFHPA
SGFAWQFSVLSRYLDPQWSIIGIQSPRPHGPMQTATNLDEVCEAHLATLLEQQPHGPYYLLGYSLGGTLA
QGIAARLRARGEQVAFLGLLDTWPPETQNWQEKEANGLDPEVLAEINREREAFLAAQQGSTSTELFTTIE
GNYADAVRLLTTAHSVPFDGKATLFVAERTLQEGMSPERAWSPWIAELDIYRQDCAHVDIISPGAFVKIG
PIIRATLNR
SEQ ID NO. 5
EntA
Amino Acid
E. Coli
MDFSGKNVWVTGAGKGIGYATALAFVEAGAKVTGFDQAFTQEQYPFATEVMDVADAGQVAQVCQRLLAET
ERLDVLINAAGILRMGATDQLSKEDWQQTFAVNVGGAFNLFQQTMNQFRRQRGGAIVTVASDAAHTPRIG
MSAYGASKAALKSLALSVGLELAGSGVRCNVVSPGSTDTDMQRTLWVSDDAEEQRIRGFGEQFKLGIPLG
KIARPQEIANTILFLASDLASHITLQDIVVDGGSTLGA
SEQ ID NO. 6
EntC
Amino Acid
E. Coli
MEDDMDTSLAEEVQQTMATLAPNREFFMSPYRSFTTSGCFARFDEPAVNGDSPDSPFQQKLAALFADAKA
QGIKNPVMVGAIPFDPRQPSSLYIPESWQSFSRQEKQTSARRFTRSQSLNVVERQAIPEQTTFEQMVARA
AALTATPQVDKVVLSRLIDITTDAAIDSGVLLERLIAQNPVSYNFHVPLADGGVLLGASPELLLRKDGER
FSSIPLAGSARRQPDEVLDREAGNRLLASEKDRHEHELVTQAMKEVLRERSSELHVPSSPQLITTPTLWH
LATPFEGKANSQENALTLACLLHPTPALSGFPHQAATQVIAELEPFDRELFGGIVGWCDSEGNGEWVVTI
RCAKLRENQVRLFAGAGIVPASSPLGEWRETGVKLSTMLNVFGLH

What is claimed is:

1. A method of treating iron-deficiency comprising administering a therapeutically effective amount of ferric enterobactin (Fe-Ent), or an Fe-Ent analog, or a pharmaceutically acceptable to salt in a subject in need thereof

2. The method of claim 1, wherein said Fe-Ent analog is selected from the group consisting of: TRENCAM, SERSAM, SER(3M)SAM, TRENSAM, and TREN(3M)SAM, wherein said Fe-Ent analogs may be complexed with iron.

3. The method of claim 1, wherein said therapeutically effective amount of Fe-Ent, or said Fe-Ent analog is isolated.

4. The method of claim 1, wherein said Fe-Ent, or Fe-Ent analog is combined with a pharmaceutically acceptable carrier.

5. The method of claim 4, wherein said pharmaceutically acceptable carrier comprises a nutritional supplement.

6. The method of claim 5, wherein said nutritional supplement comprises a probiotic bacterium configured to express a heterologous nucleotide, operably linked to a promotor, encoding one or more genes for the biosynthesis of Ent.

7. The method of claim 1, wherein said iron-deficiency comprises iron-deficiency anemia, or anemia caused by dysregulation in the DMT1 iron uptake system of said subject, anemia caused by low erythrocyte counts.

8. The method of claim 7, wherein said subject in need thereof comprises a human subject.

9. A method of treating iron-deficiency or anemia comprising administering a therapeutically effective amount of a genetically modified probiotic bacteria expressing a heterologous nucleotide, operably linked to a promotor, encoding one or more genes for the biosynthesis of enterobactin (Ent) that may be complexed with iron to form Fe-Ent.

10. The method of claim 9, wherein said probiotic bacteria comprises an Enterobacter probiotic bacterium.

11. The method of claim 9, wherein said heterologous nucleotide comprises a heterologous nucleotide encoding one or more of the genes selected from the group consisting of: entA entB, entC, entD, entE, and entF.

12. The method of claim 9, wherein said heterologous nucleotide comprises a heterologous nucleotide sequence encoding one or more of the amino acid sequences selected from the group consisting of: SEQ ID NO's. 1-6.

13. The method of claim 9, wherein said a subject in need thereof is a human subject.

14. The method of claim 13, wherein said iron-deficiency comprises iron-deficiency anemia, or anemia caused by dysregulation in the DMT1 iron uptake system of said subject.

15. A method comprising:
administering a therapeutically effective amount of ferric enterobactin (Fe-Ent), Fe-Ent analog, or a or a pharmaceutically acceptable salt to a subject in need thereof and exhibits one or more effects in said subject:
promotes mitochondrial iron uptake in said subject by the binding of said Fe-Ent, Fe-Ent analog, or a pharmaceutically acceptable salt thereof to ATP synthase α-subunit;
treats symptoms of iron-deficiency, and optionally iron-deficiency anemia in said subject;
treats symptoms of anemia caused by dysregulation in the DMT1 iron uptake system of said subject; and
promotes the production of erythrocytes in said subject.

16. The method of claim 15, wherein said Fe-Ent analog is selected from the group consisting of: TRENCAM, SERSAM, SER(3M)SAM, TRENSAM, and TREN(3M)SAM, wherein said Fe-Ent analogs may be complexed with iron.

17. A method of claim 15, wherein said therapeutically effective amount of Fe-Ent, or said Fe-Ent analog is isolated.

18. A method of claim 15, wherein said step of administering a therapeutically effective amount of Fe-Ent, or a Fe-Ent analog comprises administering a nutritional supplement having, or being configured to biosynthesize, a therapeutically effective amount of Fe-Ent that may be complexed with iron to form Fe-Ent.

19. A method of claim 17, wherein said nutritional supplement comprises a probiotic bacterium configured to express a heterologous nucleotide, operably linked to a promotor, encoding one or more of the genes selected from the group consisting of: entA entB, entC, entD, entE, and entF.


SEQUENCE LISTING (6 sequences)

SEQ ID NO: 1 - protein, 285 aa, E. Coli
MAIPKLQAYALPESHDIPQNKVDWAFEPQRAALLIHDMQDYFVSFWGENCPMMEQVIANIAALRDYCKQHNIPVYYTAQPKEQSDEDRALLNDMWGPGLTRSPEQQKVVDRLTPDADDTVLVKWRYSAFHRSPLEQMLKESGRNQLIITGVYAHIGCMTTATDAFMRDIKPFMVADALADFSRDEHLMSLKYVAGRSGRVVMTEELLPAPVPASKAALREVILPLLDESDEPFDDDNLIDYGLDSVRMMALAARWRKVHGDIDFVMLAKNPTIDAWWKLLSREVK

SEQ ID NO: 2 - protein, 207 aa, E. Coli
MRHHRTVLPLAGYTIQQIDFDPATFQPEDLFWLPYHASLTGWGRKRQAEHLAGRIAAAYALREVGEKRLPAIGDQRQPLWPTPWFGSISHCGQRALAVIADRPVGVDIERRFTPQLAAELESSIISPAEKTALLRSGLPFPLALTLAFSAKESGFKACHPDVQAGVGFNDFTLAAIKEGNLRLRLSTVEYRLQWIQAGEYIITLCAP

SEQ ID NO: 3 - protein, 536 aa, E. Coli
MSIPFTRWPEEFARRYREKGYWQDLPLTDILTRHAASDSIAVIDGERQLSYRELNQAADNLACSLRRQGIKPGETALVQLGNVAELYITFFALLKLGVAPVLALFSHQRSELNAYASQIEPALLIADRQHALFSGDDFLNTFVTEHSSIRVVQLHNDSGEHNLQDAINHPAEDFTATPSPADEVAYFQLSGGTTGTPKLIPRTHNDYYYSVRRSVEICQFTQQTRYLCAIPAAHNYAMSSPGSLGVFLAGGTVVLAADPSATLCFPLIEKHQVNVTALVPPAVSLWLQALTEGESRAQLASLKLLQVGGARLSATLAARIPAEIGCQLQQVFGMAEGLVNYTRLDDSAEKIIHTQGYPMCPDDEVWVADAEGNPLPQGEVGRLMTRGPYTFRGYYKSPQHNASAFDANGFYCSGDLISIDPEGYITVQGREKDQINRGGEKIAAEEIENLLLRHPAVIYAALVSMEDELMGEKSCAYLVVKEPLRAVQVRRFLREQGIAEFKLPDRVECVDSLPLTAVGKVDKKQLRQWLASRASA

SEQ ID NO: 4 - protein, 1339 aa, E. Coli
MAATMRLTGRLGHCVSAAVTGVLPAVAGSPLAYSDTDEFYPVAGGTMSQHLPLVAAQPGIWMAEKLSELPSAWSVAHYVELTGEVDSPLLARAVVAGLAQADTLRMRFTEDNGEVWQWVDDALTFELPEIIDLRTNIDPHGTAQALMQADLQQDLRVDSGKPLVFHQLIQVADNRWYWYQRYHHLLVDGFSFPAITRQIANIYCTWLRGEPTPASPFTPFADVVEEYQQYRESEAWQRDAAFWAEQRRQLPPPASLSPAPLPGRSASADILRLKLEFTDGEFRQLATQLSGVQRTDLALALAALWLGRLCNRMDYAAGFIFMRRLGSAALTATGPVLNVLPLGIHIAAQETLPELATRLAAQLKKMRRHQRYDAEQIVRDSGRAAGDEPLFGPVLNIKVFDYQLDIPDVQAQTHTLATGPVNDLELALFPDVHGDLSIEILANKQRYDEPTLIQHAERLKMLIAQFAADPALLCGDVDIMLPGEYAQLAQINATQVEIPETTLSALVAEQAAKTPDAPALADARYLFSYREMREQVVALANLLRERGVKPGDSVAVALPRSVFLTLALHAIVEAGAAWLPLDTGYPDDRLKMMLEDARPSLLITTDDQLPRFSDVPNLTSLCYNAPLTPQGSAPLQLSQPHHTAYIIFTSGSTGRPKGVMVGQTAIVNRLLWMQNHYPLTGEDVVAQKTPCSFDVSVWEFFWPFIAGAKLVMAEPEAHRDPLAMQQFFAEYGVTTTHFVPSMLAAFVASLTPQTARQNCATLKQVFCSGEALPADLCREWQQLTGAPLHNLYGPTEAAVDVSWYPAFGEELAQVRGSSVPIGYPVWNTGLRILDAMMHPVPPGVAGDLYLTGIQLAQGYLGRPDLTASRFIADPFAPGERMYRTGDVARWLDNGAVEYLGRSDDQLKIRGQRIELGEIDRVMQALPDVEQAVTHACVINQAAATGGDARQLVGYLVSQSGLPLDTSALQAQLRETLPPHMVPVVLLQLPQLPLSANGKLDRKALPLPELKTQASGRAPKAGSETIIAAAFASLLGCDVQDADADFFALGGHSLLAMKLAAQLSRQFARQVTPGQVMVASTVAKLATIIDGEEDSSRRMGFETILPLREGNGPTLFCFHPASGFAWQFSVLSRYLDPQWSIIGIQSPRPHGPMQTATNLDEVCEAHLATLLEQQPHGPYYLLGYSLGGTLAQGIAARLRARGEQVAFLGLLDTWPPETQNWQEKEANGLDPEVLAEINREREAFLAAQQGSTSTELFTTIEGNYADAVRLLTTAHSVPFDGKATLFVAERTLQEGMSPERAWSPWIAELDIYRQDCAHVDIISPGAFVKIGPIIRATLNR

SEQ ID NO: 5 - protein, 248 aa, E. Coli
MDFSGKNVWVTGAGKGIGYATALAFVEAGAKVTGFDQAFTQEQYPFATEVMDVADAGQVAQVCQRLLAETERLDVLINAAGILRMGATDQLSKEDWQQTFAVNVGGAFNLFQQTMNQFRRQRGGAIVTVASDAAHTPRIGMSAYGASKAALKSLALSVGLELAGSGVRCNVVSPGSTDTDMQRTLWVSDDAEEQRIRGFGEQFKLGIPLGKIARPQEIANTILFLASDLASHITLQDIVVDGGSTLGA

SEQ ID NO: 6 - protein, 395 aa, E. Coli
MEDDMDTSLAEEVQQTMATLAPNRFFFMSPYRSFTTSGCFARFDEPAVNGDSPDSPFQQKLAALFADAKAQGIKNPVMVGAIPFDPRQPSSLYIPESWQSFSRQEKQTSARRFTRSQSLNVVERQAIPEQTTFEQMVARAAALTATPQVDKVVLSRLIDITTDAAIDSGVLLERLIAQNPVSYNFHVPLADGGVLLGASPELLLRKDGERFSSIPLAGSARRQPDEVLDREAGNRLLASEKDRHEHELVTQAMKEVLRERSSELHVPSSPQLITTPTLWHLATPFEGKANSQENALTLACLLHPTPALSGFPHQAATQVIAELEPFDRELFGGIVGWCDSEGNGEWVVTIRCAKLRENQVRLFAGAGIVPASSPLGEWRETGVKLSTMLNVFGLH